SEQUENCE LISTING (15 sequences)

SEQ ID NO: 1 - DNA, 156 bp, Homo sapiens
  source 1..156: mol_type = other DNA
ggtaccttttgatttctctatctcagcagaaccttctaagtctcagacacgtaaacccaagtgtggcaaaggaactcattgctctcgaaatgcatatatgttggtttatagactgcaaactcaagaaaagcccaacactactgttcaagttccagg

SEQ ID NO: 2 - protein, 43 aa, Homo sapiens
AEPSKSQTRKPKCGKGTHCSRNAYMLVYRLQTQEKPNTTVQVP

SEQ ID NO: 3 - DNA, 29 bp, synthetic construct
  source 1..29: mol_type = other DNA
tgctctcgaaatgcatatgtgctagttta

SEQ ID NO: 4 - DNA, 19 bp, Homo sapiens
  source 1..19: mol_type = other DNA
tctcgaaatgcatatatgt

SEQ ID NO: 5 - DNA, 29 bp, synthetic construct
  source 1..29: mol_type = other DNA
tgctctcgaaatgcatatatactagttta

SEQ ID NO: 6 - DNA, 148 bp, Homo sapiens
  source 1..148: mol_type = other DNA
ccctccaactcataaagccaagccacagattcctgctgagcgggatagggaaccttccaaactgaagcgctcctactcctccccagatataacccaggctattcaagaggaagagaagaggaagccaacagtaactccaacagttaat

SEQ ID NO: 7 - protein, 49 aa, Homo sapiens
PPTHKAKPQIPAERDREPSKLKRSYSSPDITQAIQEEEKRKPTVTPTVN

SEQ ID NO: 8 - DNA, 33 bp, synthetic construct
  source 1..33: mol_type = other DNA
accttcgaaactgaagcgctcctgcccctcccc

SEQ ID NO: 9 - DNA, 19 bp, synthetic construct
  source 1..19: mol_type = other DNA
gagtaggagcgcttcagtt

SEQ ID NO: 10 - DNA, 25 bp, synthetic construct
  source 1..25: mol_type = other DNA
ttgaagtttatcgccattttattcg

SEQ ID NO: 11 - DNA, 22 bp, synthetic construct
  source 1..22: mol_type = other DNA
gctggtatagccatccacagaa

SEQ ID NO: 12 - DNA, 23 bp, synthetic construct
  source 1..23: mol_type = other DNA
tgttgttctaggtttgttcccca

SEQ ID NO: 13 - DNA, 23 bp, synthetic construct
  source 1..23: mol_type = other DNA
tcaccaacattcaccttatggaa

SEQ ID NO: 14 - protein, 14 aa, synthetic construct
  DISULFID 3..14: note = Disulfide bridge
AGCKNFFWKTFTSC

SEQ ID NO: 15 - protein, 28 aa, synthetic construct
  DISULFID 17..28: note = Disulfide bridge
SANSNPAMAPRERKAGCKNFFWKTFTSC

(19) United States
(12) Patent Application Publication
(10) Pub. No.: US 2026/0325504 A1
(43) Pub. Date: Oct. 22, 2026

(54) Genetically Engineered Human Pituitary Corticotroph Tumor Organoids Exhibit Divergent Responses to Selective Glucocorticoid Receptor Modulators

(71) Applicant: Corcept Therapeutics Incorporated, Redwood City, CA (US)

(72) Inventors: Andrew Greenstein, San Francisco, CA (US); Andreas Moraitis, Sunny Isles Beach, FL (US); Yana Zavros, Tucson, AZ (US)

(73) Assignee: Corcept Therapeutics Incorporated, Redwood City, CA (US)

(21) Appl. No.: 19/109,395

(22) PCT Filed: Sep. 1, 2023
(86) PCT No.: PCT/US2023/073348

§ 371 Date: Mar. 6, 2025

Related U.S. Application Data
(60) Provisional application No. 63/404,130, filed on Sep. 6, 2022.

(51) Int. Cl. A61K 31/4745; A61K 31/407; A61P 5/00

(52) U.S. Cl. CPC A61K 31/4745; A61K 31/407; A61P 5/00

(57) ABSTRACT

Provided herein is a method for determining selectively increasing the expression levels of SSTR5 in pituitary tumor cells in a patient, the method comprising administering to the patient a glucocorticoid receptor modulator (GRM) to the patient, wherein the GRM is capable of selectively stimulating the expression of SSTR5. Also provided are methods of inducing apoptosis in pituitary tumor cells or treating a pituitary tumor in a patient without resulting in significantly increasing the level of ACTH and methods of detecting a pituitary tumor by administering the GRM.

### RELATED APPLICATION

This application claims priority to, and the benefit of, U.S. Provisional Application No. 63/404,130, filed on Sep. 6, 2022. The entire content of the provisional application is herein incorporated by reference for all purposes.

### REFERENCE TO A SEQUENCE LYSING

This application incorporates by reference the Sequence Listing submitted herewith. The Sequence Listing.xml file, identified as 085178-1399969-017710WO, is 15,497 bytes in size and was created on Sep. 1, 2023.

### BACKGROUND

Cushing disease (CD) is a serious endocrine disorder that is caused by an adrenocorticotropic hormone (ACTH)-secreting pituitary neuroendocrine tumor (PitNETs) that leads to excess adrenal cortisol secretion [1-4]. Renamed as PitNETs by the WHO [5], pituitary tumors have been defined as benign, implying that these tumors cause a disease that is not life threatening or harmful to health. However, chronic exposure to excess and unregulated cortisol has wide ranging and detrimental effects on health, including increased stroke rates, diabetes, obesity, depression, anxiety and a threefold increase in the risk of death from cardiovascular disease and cancer [4, 6, 7, 8]. The first-line treatment for CD is pituitary surgery, which is followed by disease remission in only 56% of patients during the 10-year follow-up period post-surgery, even in the hands of an experienced surgeon [9]. Studies have demonstrated that surgical failures and late recurrences of CD are common, and despite multiple treatment regimens, biochemical control still fails in approximately 30% of patients, suggesting that in routine clinical practice, initial and long-term remission is not achieved in a substantial number of patients with CD [7, 10].

Medical therapy is often considered when initial surgery fails to achieve remission, or when the tumor recurs after apparent surgical remission. Approved treatments include pituitary-targeted drugs, adrenal steroidogenic inhibitors, and a non-selective glucocorticoid receptor (GR) antagonist [3, 11, 12]. There is an inverse relationship between disease duration and reversibility of complications associated with the disease, thus emphasizing the importance of identifying an effective medical strategy to rapidly normalize hypercortisolemia/cortisol activity by directly targeting the pituitary adenoma [4, 7, 10]. Standard of care treatments have low efficacy and/or tolerability, making CD a medical therapeutic challenge. For example, mifepristone (Korlym®), a non-selective GR antagonist approved by the Food and Drug Administration in 2012 for the treatment of hyperglycemia secondary to endogenous Cushing's syndrome, including CD, in adults who have failed surgery or are not candidates for surgery, competes with cortisol binding at the GR and significantly improves hyperglycemia secondary to hypercortisolism in patients with CD [13]. However, while mifepristone improves the symptoms of CD, it also raises ACTH (and thus cortisol) levels and exhibits significant side effects in some patients. Through its binding to the progesterone receptor, mifepristone causes side effects including endometrial thickening and vaginal bleeding [13, 14]. Relacorilant is an investigational, selective GR modulator that, unlike mifepristone, has no detectable binding to the progesterone, estrogen and androgen receptors [15, 16]. In a phase 2 study in patients with Cushing's syndrome, relacorilant reversed the effects of excess cortisol on hypertension and insulin resistance [16], did not significantly raise ACTH or cortisol levels, and has been reported to induce tumor regression in some CD patients with pituitary macroadenomas [17, 18]. Relacorilant is currently under investigation in phase 3 trials for the treatment of patients with endogenous Cushing syndrome (GRACE (NCT03697109) and GRADIENT (NCT04308590) studies). The mechanisms driving relacorilant-induced tumor regression, and the role of tumor sensitization to endogenous somatostatin, remain unknown due to the lack of advanced multicellular in vitro models that recapitulate the complexity of the PitNET microenvironment. The absence of preclinical models that recapitulate the tumor tissue has prevented us from acquiring the knowledge to develop and implement therapies specifically targeted the tumor with a higher efficacy and tolerability for CD patients.

### BRIEF DESCRIPTION OF THE DRAWINGS

FIGS. 1A and 1B show the generation of pituitary tumor organoids from iPSCs or adenoma tissue. (A) Differentiation schedule for the generation of pituitary organoids derived from Induced Pluripotent Stem Cells (IPSCs) expressing somatic mutations driving the development of CD. Pituitary organoids were also generated based on the outlined schedule using iPSCs generated from Peripheral Blood Mononuclear Cells (PBMCs) collected from a patient with CD, or a healthy individual. Bright field images demonstrating morphological variation is observed between iPSC^{ctrl} and iPSC^{USP48} or iPSC^{USP8} lines and organoids. (B) Overview of the generation of Human Pituitary Organoids (hPITOs) from adenoma tissues collected from patients with CD.

FIGS. 2A-2D show the expression pattern of pituitary hormone-producing cell lineages in iPSCs differentiated to pituitary organoids. Expression of PIT-1, ACTH, Growth Hormone (GH), Follicle-Stimulating Hormone (FSH), LH, Prolactin (PRL) and synaptophysin (synapto) with co-stain Hoechst (nuclei) was measured by immunofluorescence using chamber slides collected at day 15 (D15) of the differentiation schedule of (FIG. 2A) control iPSCs (iPSC^{ctrl}) and iPSCs expressing (FIG. 2B) USP48M415V (iPSC^{USP8SMV}), (FIG. 2C) USP48M415I (iPSC^{USP48MI}) and (FIG. 2D) USP8 (iPSC^{USP8}) mutations. Arrows highlight the increased expression of ACTH and synaptophysin with the concomitant loss of Pituitary-specific positive transcription factor 1 (PIT1), GH, FSH, LH and PRL in iPCSs expressing somatic mutations USP48 and USP8. Quantification of the percentage of positive cells over the total cells (Y axis) is shown in the dot plots for each iPSC line.

FIGS. 3A-3D show the expression of cell lineages and transcription factors in iPSC generated pituitary tumor organoids. (FIG. 3A) Immunohistochemistry using FFPE sections prepared from iPSC^{ctrl}, iPSC^{USP48MV}, iPSC^{USP48MI}, and iPSC^{USP8} organoids stained with antibodies specific for CAM5.2, T-Pit, Synaptopbysin and ACTH. (FIG. 3B) viSNE maps showing concatenated flow cytometry standard files for iPSC^{ctrl}, iPSC^{USP48MV}, iPSC^{USP48MI} M, and iPSC^{USP8} organoids 30 days post-directed differentiation. Maps define spatially distinct cell populations using pituitary specific cell lineage, stem cell and transcription factor markers between iPSC^{ctrl} organoids and mutant lines. (FIG. 3C) Immunofluorescence staining of ACTH, PIT1 and LH expression in iPSC^{ctrl} and iPSC^{USP48MV} cultures 15 days post-directed differentiation. (FIG. 3D) Differential expression of transcription factors T-Box Transcription Factor 19 (TPit), PIT1 and SF1 and POMC were measured by qRT-PCR. * p<0.05 compared to iPSC^{ctrl} organoids, n=3 individual experimental replicates.

FIGS. 4A-4C show proliferation and ACTH secretion during directed differentiation of IPSC organoids. (FIG. 4A) An ELISA was performed using conditioned media collected during the differentiation schedule from iPSC^{ctrl} and iPSC mutant cultures for the measurement of ACTH secretion (pg/ml). (FIG. 4B) Immunofluorescence images of expression of EdU in iPSC^{ctrl}, iPSC^{USP8SMI}, iPSC^{USP48MV}, and iPSC^{USP8} organoids. (FIG. 4C) Quantification of EdU positive cells of iPSC^{ctrl} and mutant organoids. * p<0.05, *** p<0.0001 compared to iPSC^{ctrl} organoids, n=4 individual organoids quantified per culture.

FIGS. 5A-5D show differential expression of SSTR2 and SSTR5 in iPSC generated pituitary tumor organoids in response to mifepristone and relacorilant. Differential expression of (FIG. 5A) SSTR2 and (FIG. 5B) SSTR5 in iPSC^{ctrl}, iPSC^{USP48MI}, iPSC^{USP48MV} and iPSC^{USP8} organoids in response to vehicle (Veh), mifepristone (Mife) or relacorilant (Rela). *p<0.05 compared to vehicle-treated organoids, #p<0.05 compared to mifepristone treated organoids, n=3 experimental replicates/organoid line. (FIG. 5C) Differential expression of SSTR2 and SSTR5 in iPSC^{ctrl} organoids treated with vehicle (Veh), mifepristone (Mife), GANT61 (GANT), Mife+GANT, ketoconazole (Keto), Mife+Keto, or dexamethasone (Dexa). * p<0.05 compared to iPSCcrtl organoids treated with Veh, n=3 experimental replicates/organoid line. (FIG. 5D) Differential expression of SSTR2 and SSTR5 in iPSCcrtl organoids treated with vehicle (Veh), relacorilant (Rela), GANT61 (GANT), Rela+GANT, ketoconazole (Keto), Rela+Keto, or dexamethasone (Dexa), *p<0.05 compared to iPSCcrtl organoids treated with Veh, n=3 experimental replicates/organoid line.

FIGS. 6A-6G show changes in pituitary tumor cell proliferation, viability, and ACTH secretion in iPSC^{ctrl} organoids in response to mifepristone and relacorilant. (FIG. 6A) Immunofluorescence images of EdU expression in iPSC^{ctrl} organoids in response to vehicle (Veh), mifepristone (Mife), pasireotide (Pas), octreotide (Oct), relacorilant (Rela), Mife+Pas, Mife+Oct, Rela+Pas, Rela+Oct, dexamethasone (Dexa), (FIG. 6B) Quantification of EdU positive cells of iPSC^{ctrl} and mutant organoids. * p<0.05 compared to iPSC^{ctrl} organoids, n=4 individual organoids quantified per culture. (FIG. 6C) Representative Hoescht staining of iPSC^{ctrl} organoids in response to experimental treatments for the calculation of nuclear irregularity index (NII). Nuclear morphometric analysis of iPSC^{ctrl} organoids in response to experimental treatments with quantification shown for (FIG. 6D) Veh, (FIG. 6E) mifepristone, or (FIG. 6F) relacorilant treatments. Morphometric classification of NII was based on the normal (N), small(S), small regular (SR), short irregular (SI), large regular (LR), large irregular (LI) and irregular (I) nuclear morphology. (FIG. 6G) An ELISA was performed using conditioned media collected from iPSC^{ctrl} cultures in response to treatments for the measurement of ACTH secretion (pg/ml). * p<0.05 compared to Veh treatment, #p<0.05 compared to mifepristone or relacorilant alone, n=4 individual organoids quantified per culture.

FIG. 7A-7G show changes in pituitary tumor cell proliferation, viability and ACTH secretion in iPSC^{USP48MV} organoids in response to mifepristone and relacorilant. (FIG. 7A) Immunofluorescence images of EdU expression in iPSC^{USP48MV} organoids in response to vehicle (Veh), mifepristone (Mife), pasireotide (Pas), octreotide (Oct), relacorilant (Rela), Mife+Pas, Mife+Oct, Rela+Pas, Rela+Oct, dexamethasone (Dexa). (FIG. 7B) Quantification of EdU positive cells of iPSC^{USP48MV} and mutant organoids. * p<0.05 compared to iPSC^{USP48MV} organoids, n=4 individual organoids quantified per culture. (FIG. 7C) Representative Hoescht staining of iPSC^{ctrl} organoids in response to experimental treatments for the calculation of nuclear irregularity index (NII). Nuclear morphometric analysis of iPSC^{USP48MV} organoids in response to experimental treatments with quantification shown for (FIG. 7D) Veh, (FIG. 7E) mifepristone, or (FIG. 7F) relacorilant treatments. Morphometric classification of NII was based on the normal (N), small(S), small regular (SR), small irregular (SI), large regular (LR), large irregular (LI) and irregular (I) nuclear morphology. (FIG. 7G) An ELISA was performed using conditioned media collected from iPSC^{USP48MV} cultures in response to treatments for the measurement of ACTH secretion (pg/ml). * p<0.05 compared to Veh treatment, #p<0.05 compared to mifepristone or relacorilant alone, n=4 individual organoids quantified per culture.

FIGS. 8A-8H show changes in SSTR2, SSTR5 and ACTH expression, and pituitary tumor cell death in iPSC^{USP48MV} organoids in response to mifepristone and relacorilant. Fluorescent intensity of (FIG. 8A) SSTR2, (FIG. 8B) SSTR5, (FIG. 8C) ACTH, and (FIG. 8D) zombie of viSNE heatmaps for iPSC^{USP48MV} organoids in response to vehicle (Veh), pasireotide (Pas), relacorilant (Rela), Rela+Pas, mifepristone (Mife), and Mife+Pas. iPSC^{USP48}=15000 events. Quantification of percentage of SSTR2 (E), SSTR5 (F), ACTH (G), or Zombie (H) positive cells within iPSC^{USP48MV} organoid cultures in response to experimental treatments. *p<0.05 compared to Veh.

FIGS. 9A-9L show changes in SSTR2, SSTR5 and ACTH expression, and pituitary tumor cell death in hPITOs in response to mifepristone and relacorilant. (FIG. 9A) Brightfield images of organoid cultures generated from patients with CD (hPITOs 37, 38, 39, 40). (FIG. 9B) Immunofluorescence staining using antibodies specific for CAM5.2 (upper left panel) and ACTH (upper right panel) of hPITO37. (FIG. 9C) viSNE maps showing concatenated flow cytometry standard files for hPITO37 line defining the spatially distinct cell populations using pituitary specific cell lineages, stem cell and transcription factor markers. (FIG. 9D) Differential expression of SSTR2 and SSTR5 measured in hPITO36, 37, 38, 39 and 40 lines. Fluorescent intensity of (FIGS. 9E, 9F) SSTR2, (FIGS. 9G, 9H) SSTR5, (FIGS. 9I, 9J) ACTH, and (FIGS. 9K, 9L) zombie of viSNE heatmaps for iPSC^{USP48MV} organoids in response to vehicle (Veh), pasireotide (Pas), relacorilant (Rela), Rela+Pas, mifepristone (Mife), and Mife+Pas. hPITO=5000 events. Quantification of percentage of SSTR2, SSTR5, ACTH, or Zombie positive cells within hPITO cultures in response to experimental treatments is shown in F, H, J and L. * p<0.05 compared to Veh.

FIG. 10: Summary of divergent effects of mifepristone and relacorilant on SSTR2 and SSTR5 expression, ACTH secretion, and pituitary tumor proliferation and apoptosis. Mifepristone induces SSTR2 with greater magnitude compared to SSTR5. Relacorilant predominantly induces SSTR5 and tumor cell apoptosis. GLI family zinc finger1 (GLI) signaling inhibitor GANT61 blocks SSTR2 and SSTR5 expression in response to mifepristone and relacorilant. SMO inhibitor ketoconozaole has no effect on the induction of SSTR2 and SSTR5 by GR modulators mifepristone and relacorilant. GR antagonists are likely to signal via a non-canonical GLI1 SMO-independent signaling pathway. Arrows indicate the magnitude of induction.

FIGS. 11A-11C show CRISPR mutation design and validation of gene edited iPSC generated organoids. (FIG. 11A) Design of somatic mutations associated with CD, including Ubiquitin carboxyl-terminal hydrolase 8 (USP8), Ubiquitin carboxyl-terminal hydrolase 48 (USP48), and BRAF) in corticotroph pituitary tumors. Restriction Fragment Length Polymorphism (RFLP) validation was used to confirm successful gene editing. Undigested (U) DNA (FIG. 11B) were compared to samples digested with the restriction enzyme (D). Respective restriction enzymes for each mutation were used (AvaII for BRAF, SpeI for USP48, BstBI for USP8). (FIG. 11C) Arrows indicate the U and D bands on the gel. iPSC^{ctrl} displayed no separation of bands in digested versus undigested samples or amplification of the mutated genes. FIG. 11 contains SEQ ID NOs 1-9.

FIGS. 12A-12F show drug responses of hPITOs and adjacent normal pituitary organoids. Dose-response curves using hPITO36-40 and hPITO37N and 38N generated from adjacent normal pituitary tissue in response (FIG. 12A, 12B) pasireotide, (FIGS. 12C, 12D) mifepristone, and (E, F) relacorilant.

FIGS. 13A-13D show the expression pattern of major hormone-producing cell lineages in iPSCs differentiated to pituitary organoids. Expression of PIT-1, ACTH, GH, FSH, LH, PRL and synaptophysin (synapto) with co-stain Hoechst (nuclei) was measured by immunofluorescence using chamber slides collected at day 4 (D4) of the differentiation schedule of (FIG. 13A) control iPSCs (iPSC^{ctrl}) and iPSCs expressing (FIG. 13B) USP48MV (IPSC^{USP48MV}) (FIG. 13C) USP48MI (iPSC^{USP48MI}) and (FIG. 13D) USP8 (iPSC^{USP8}) mutations. Arrows highlight the increased expression of ACTH and synaptophysin with the concomitant loss of PIT1, GH, FSH, LH and PRL in iPCSs expressing somatic mutations USP48 and USP8. Quantification of the percentage of positive cells over total cells (Y axis) is shown in the dot plots for each iPSC line.

FIG. 14 shows the results of nuclear morphometric analysis of iPSC^{ctrl} organoid cultures in response to mifepristone and relacorilant. Morphometric classification of NII was based on the normal (N), small(S), small regular (SR), short irregular (SI), large regular (LR), large irregular (LI) and irregular (I) nuclear morphology. Distribution of nuclear morphology in response to in response to vehicle (Veh), mifepristone (Mife), pasireotide (Pas), octreotide (Oct), relacorilant (Rela), Mife+Pas, Mife+Oct, Rela+Pas, Rela+Oct, dexamethasone (Dexa) was plotted.

FIG. 15 shows the results of nuclear morphometric analysis of iPSC^{USP48MV} organoid cultures in response to mifepristone and relacorilant. Morphometric classification of NII was based on the normal (N), small(S), small regular (SR), short irregular (SI), large regular (LR), large irregular (LI) and irregular (I) nuclear morphology. Distribution of nuclear morphology in response to in response to vehicle (Veh), mifepristone (Mife), pasireotide (Pas), octreotide (Oct), relacorilant (Rela), Mife+Pas, Mife+Oct, Rela+Pas, Rela+Oct, dexamethasone (Dexa) was plotted.

FIGS. 16A-16C show changes in FKBP5 expression in response to mifepristone, relacorilant and dexamethasone. Quantitative RT-PCR was performed on RNA extracted from (FIG. 16A) iPSC^{ctrl}, (FIG. 16B) iPSC^{USP48MV}, and (FIG. 16C) IPSC^{USP8}, organoids treated with vehicle (Veh), mifepristone (Mife), relacorilant (Rela) or dexamethasone (Dexa).

### BRIEF SUMMARY

In one aspect, provided herein is a method of selectively increasing SSTR5 expression in human pituitary cells in a patient, the method comprising administering to the patient a selective glucocorticoid receptor modulator (SGRM) to the patient, wherein the SGRM is capable of selectively stimulating the expression of SSTR5.

In another aspect, disclosed herein is a method of inducing apoptosis in pituitary tumor cells or treating a pituitary tumor in a patient without resulting in significantly increasing the level of ACTH, the method comprising administering a SGRM to the patient, wherein the SGRM is capable of selectively stimulating the expression of SSTR5.

In another aspect, disclosed herein a method of detecting a pituitary tumor in a patient, the method comprising administering a SGRM to the patient, wherein the SGRM selectively stimulates the expression of SSTR5 in the pituitary tumor cells and administering an agent that selectively binds to the SSTR5, and the agent that bind to SSTR5.

In another aspect, disclosed herein is a use of selective glucocorticoid receptor modulator (SGRM) for the treatment of Cushing's syndrome by the selective increase of SSTR5 expression, wherein the SGRM is capable of selectively stimulating the expression of SSTR5. In another aspect, disclosed herein is a use of of a selective glucocorticoid receptor modulator (SGRM) in the manufacture of a medicament for treating Cushing's syndrome by the selective increase of SSTR5 expression, wherein the SGRM is capable of selectively stimulating the expression of SSTR5.

In another aspect, disclosed herein is the use of a selective glucocorticoid receptor modulator (SGRM) for the treatment of Cushing's syndrome by the selective increase of SSTR5 expression, wherein the SGRM is capable of selectively stimulating the expression of SSTR5.

In another aspect, disclosed herein is the use of a selective glucocorticoid receptor modulator (SGRM) in the manufacture of a medicament for treating Cushing's syndrome by the selective increase of SSTR5 expression, wherein the SGRM is capable of selectively stimulating the expression of SSTR5.

In another aspect, disclosed herein the use of a selective glucocorticoid receptor modulator (SGRM) for treating a pituitary tumor without significantly increasing the level of ACTH, by the selective increase of SSTR5 expression, wherein the SGRM is capable of selectively stimulating the expression of SSTR5.

In another aspect, disclosed herein is the use of a selective glucocorticoid receptor modulator (SGRM) in the manufacture of a medicament for treating a pituitary tumor without significantly increasing the level of ACTH, by the selective increase of SSTR5 expression, wherein the SGRM is capable of selectively stimulating the expression of SSTR5.

In another aspect, disclosed herein is the use of a selective glucocorticoid receptor modulator (SGRM) for inducing apoptosis in a pituitary tumor without significantly increasing the level of ACTH, by the selective increase of SSTR5 expression, wherein the SGRM is capable of selectively stimulating the expression of SSTR5.

In another aspect, disclosed herein is the use of a selective glucocorticoid receptor modulator (SGRM) in the manufacture of a medicament for inducing apoptosis in a pituitary tumor without significantly increasing the level of ACTH, by the selective increase of SSTR5 expression, wherein the SGRM is capable of selectively stimulating the expression of SSTR5.

In another aspect, disclosed herein is the use of a selective glucocorticoid receptor modulator (SGRM) for detecting a pituitary tumor in a patient, wherein the SGRM selectively stimulates the expression of SSTR5 in the pituitary tumor cells, wherein said use comprises use with an agent that selectively binds to the SSTR5.

In another aspect, disclosed herein is the use of a selective glucocorticoid receptor modulator (SGRM), wherein the SGRM is capable of selectively stimulating the expression of SSTR5, for detecting a pituitary tumor in a patient.

Other objects, features, and advantages of the present disclosure will be apparent to one of skill in the art from the following detailed description and figures.

### DETAILED DESCRIPTION

### 1. Definitions

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

“Patient,” “individual” or “subject” is used interchangeably to refer to a human subject. In some cases, the individual is suspected of having a pituitary tumor.

The term “administering” includes oral administration, topical contact, administration as a suppository, intravenous, intraperitoneal, intramuscular, intralesional, intrathecal, intranasal, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, epicutaneous, subcutaneous, intraperitoneal, intraventricular, and intracranial. Other modes of delivery include, but are not limited to, the use of liposomal formulations, intravenous infusion, transdermal patches, etc.

The terms “Treat”, “treating,” and “treatment” refer to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, histopathological examination (e.g., analysis of biopsied tissue), or laboratory analysis of serum, plasma, or blood.

The term “sample” refers to a biological sample obtained from a human subject. The sample can be any cell, tissue or fluid sample from a human subject. Samples can be subject to various treatment, storage or processing procedures before being analyzed according to the methods described herein. Generally, the terms “sample” or “samples” are not intended to be limited by their source, origin, manner of procurement, treatment, processing, storage or analysis, or any modification.

The term “cortisol” refers to a glucocorticoid hormone that is produced by the zona fasciculata of the adrenal gland.

The term “adrenocorticotropic hormone” or “ACTH” refers to a polypeptide-based hormone that is normally produced and secreted by the anterior pituitary gland. ACTH stimulates secretion of cortisol and other glucocorticoids (GCs) by specialized cells of the adrenal cortex. In healthy mammals, ACTH secretion is tightly regulated. ACTH secretion is positively regulated by corticotropin releasing hormone (CRH), which is released by the hypothalamus. ACTH secretion is negatively regulated by cortisol and other glucocorticoids.

An “increase” or a “decrease” refers to a detectable positive or negative change in quantity from a comparison control. An increase is a positive change that is typically at least 5%, at least 10%, or at least 20%, 30%, 40%, or 50%, or 100%, and can be as high as at least 1.5-fold, at least 2-fold, at least 5-fold or even 10-fold of the control value. Similarly, a decrease is a negative change that is typically at least 5%, at least 10%, or at least 20%, 30%, or 50%, or even as high as at least 80% or 90% of the control value. Other terms indicating quantitative changes or differences from a comparative basis, such as “more,” “less,” ‘higher,” and “lower,” are used in this application in the same fashion as described above.

The term “significant increase” refers to an increase, as compared to the control value, that is significant (by a standard non-parametric t-test) with a p value <0.05.

The term “selective increase” or “selectively stimulate,” in the context of SSTR expression, refers to increasing expression of one SSTR to an extent that is significantly higher, for example, at least 20%, 30%, 40%, or 50%, or 100%, at least 1.5-fold, at least 2-fold, at least 5-fold or even 10-fold higher than increasing another SSTR. In one exemplary embodiment, administration of relacorilant can selectively increase SSTR5 expression to an extent that is at least at least 20%, 30%, 40%, or 50%, or 100%, at least 1.5-fold, at least 2-fold, at least 5-fold or even 10-fold higher than the increase in SSTR2 expression under the same assay conditions.

The term “pituitary tumor” as used herein includes, but is not limited to, lactotrophic adenoma or prolactinoma, ACTH-secreting adenoma, somatotrophic adenomas, corticotrophic adenoma, gonadotrophic adenoma, thyrotrophic adenomas, and null cell adenoma. An ACTH-secreting pituitary tumor may be found in the anterior lobe of the pituitary, usually measuring less than about 10 millimeters (mm) in diameter (or, where irregular in shape, less than about 10 mm in their largest linear dimension (the straight line drawn so as to measure the largest extent of the irregular shape). Most pituitary ACTH-secreting adenomas are small in size (i.e., microadenomas). The present methods are suitable for treating macroadenomas (having a linear dimension greater than 10 mm) and for treating microadenomas (smaller than 10 mm in their largest dimension). One type of pituitary tumor is a pituitary neuroendocrine tumor (PitNETs).

As used herein, the terms “somatostatin” and “SST” refers to the peptide hormone somatostatin and active variants thereof. Somatostatin has at least two naturally occurring active forms: a 14 amino acid form, and a 28 amino acid form. Alternative forms are produced by alternate cleavage of the single preproprotein encoded by this gene. Somatostatin is expressed throughout the body and inhibits the release of numerous secondary hormones by binding to high-affinity G-protein-coupled somatostatin receptors. The fourteen residue form is reported to have the following sequence: Ala-Gly-Cys-Lys-Asn-Phe-Phe-Trp-Lys-Thr-Phe-The-Ser-Cys (SEQ ID NO:14), and to have a disulfide bridge between the two cysteine residues at positions 3 and 14. The twenty-eight residue form is reported to have the following sequence: Ser-Ala-Asn-Ser-Asn-Pro-Ala-Met-Ala-Pro-Arg-Glu-Arg-Lys-Ala-Gly-Cys-Lys-Asn-Phe-Phe-Trp-Lys-Thr-Phe-Thr-Ser-Cys (SEQ ID NO:15) (Disulfide bridge: 17-28). These sequences are reported, for example, by Shen et al., PNAS 79 (150:4575-4579 (1982).

As used herein, the term “somatostatin analog” refers to a peptide or another molecule that is active at a somatostatin receptor; as used herein, a somatostatin analog binds to a somatostatin receptor. A somatostatin analog may be a somatostatin receptor agonist (i.e., it may activate a somatostatin receptor), may have partial agonist effects on a somatostatin receptor, may be a partial or may be a strong a somatostatin receptor antagonist, or have other effects when bound to, or in otherwise near to or in contact with a somatostatin receptor. Somatostatin analogs include, without limitation, octreotide, octreotate, pasireotide, lanreotide, pentetreotide, vapreotide, seglitide, cortistatin, and analogs and derivatives thereof. Somatostatin analogs are somatostatin receptor ligands.

As used herein, the term somatostatin receptor refers to a class of G-protein coupled seven transmembrane receptors that bind somatostatin. There are five somatostatin receptor sub-types, referred to as SSTR1-SSTR5 respectively. See, e.g., Trends Pharmacol Sci. 1995 March;16 (3): 86-8. The full-length human somatostatin receptors include: Type 1, with 391 amino acid residues, NCBI Accession No.: NP_001040.1; Type 2, with 369 amino acid residues, NCBI Accession No.: NP_001041.1; Type 3, with 418 amino acid residues, NCBI Accession No.: NP_001265616.1; Type 4, with 388 amino acid residues, NCBI Accession No.: NP_001043.2; and Type 5, with 364 amino acid residues, NCBI Accession No.: NP_001166031.1.

As used herein, the term “somatostatin receptor ligand,” or “somatostatin or somatostatin analog” refers to any ligand of any one of the somatostatin receptor subtypes (SSTR1-SSTR5). In some cases, the ligand is somatostatin. In some cases, the ligand is a somatostatin analog.

“Glucocorticosteroid” (“GC”) or “glucocorticoid” refers to a steroid hormone that binds to a glucocorticoid receptor. Glucocorticosteroids are typically characterized by having 21 carbon atoms, an α,β-unsaturated ketone in ring A, and an α-ketol group attached to ring D. They differ in the extent of oxygenation or hydroxylation at C-11, C-17 and C-19 (Rawn, “Biosynthesis and Transport of Membrane Lipids and Formation of Cholesterol Derivatives,” in Biochemistry, Daisy et al. (eds.), 1989, pg. 567).

“Glucocorticoid receptor” (“GR”) refers to the type II GR which specifically binds to cortisol and/or cortisol analogs such as dexamethasone (See, e.g., Turner & Muller, J Mol Endocrinol, 2005 35 283-292). The GR is also referred to as the cortisol receptor. The term includes isoforms of GR, recombinant GR and mutated GR. Inhibition constants (Kᵢ) against the human GR receptor type II (Genbank: P04150) are between 0.0001 nM to 1,000 nM; preferably between 0.0005 nM to 10 nM, and most preferably between 0.001 nM to 1 nM.

As used herein, the term “selective glucocorticoid receptor modulator” or “SGRM” refers to any composition or compound which binds to GR and modulates any biological response associated with the binding of a GR to an agonist. By “selective,” the drug preferentially binds to the GR rather than other nuclear receptors, such as the progesterone receptor (PR), the mineralocorticoid receptor (MR) or the androgen receptor (AR). Methods for determining if a compound is a SGRM are disclosed in U.S. Pat. No. 9,943,505B2, the entire disclosure of which is herein incorporated by reference.

As used herein, the term “mifepristone” refers to 11β-(4-dimethylaminophenyl)-17ß-hydroxy-17α-(1-propynyl)-estra-4,9-dien-3-one), also referred to as RU486, or as RU38.486, or as 17-beta-hydroxy-11-beta-(4-dimethyl-aminophenyl)-17-alpha-(1-propynyl)-estra-4,9-dien-3-one). Mifepristone binds to the glucocorticoid receptor (GR), typically with high affinity, and inhibits the biological effects initiated/mediated by the binding of any cortisol or cortisol analog to a GR receptor. Salts, hydrates and prodrugs of mifepristone are all included in the term “mifepristone” as used herein. Thus, used herein, “mifepristone” refers to the molecule that has the following structure:

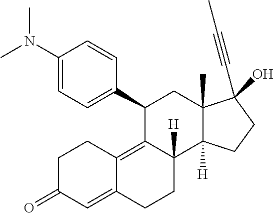

and to salts, hydrates, and prodrugs thereof, and pharmaceutical compositions thereof.

The term “Cushing's syndrome” refers to a disease caused by prolonged exposure to endogenous or exogenous glucocorticoids. Cushing's syndrome patients often suffer hyperglycemia secondary to hypercortisolism. Symptoms of Cushing's syndrome include but are not limited to one or more of the following: weight gain, high blood pressure, poor short-term memory, poor concentration, irritability, excess hair growth, impaired immunological function, ruddy complexion, extra fat in the neck region, moon face, fatigue, red stretch marks, irregular menstruation, or a combination thereof. Symptoms of Cushing's syndrome can additionally or alternatively include without limitation one or more of the following: insomnia, recurrent infection, thin skin, easy bruising, weak bones, acne, balding, depression, hip or shoulder weakness, swelling of the extremities, diabetes mellitus, elevated white blood cell count, hypokalemic metabolic alkalosis, or a combination thereof.

“Glucocorticoid receptor modulator” refers to any compound which inhibits any biological response associated with the binding of GR to an agonist. For example, a GR agonist, such as dexamethasone, increases the activity of tyrosine aminotransferase (TAT) in HepG2 cells (a human liver hepatocellular carcinoma cell line; ECACC, UK). Accordingly, GR modulators of the present disclosure can be identified by measuring the ability of the compound to inhibit the effect of dexamethasone. TAT activity can be measured as outlined in the literature by A. Ali et al., J. Med. Chem., 2004, 47, 2441-2452. In one example, a modulator is a compound with an IC₅₀ (half maximal inhibition concentration) of less than 10 micromolar.

The term “steroidal backbone” in the context of glucocorticoid receptor modulators containing such refers to glucocorticoid receptor modulators that contain modifications of the basic structure of cortisol, an endogenous steroidal glucocorticoid receptor ligand. The basic structure of a steroidal backbone is provided as Formula I:

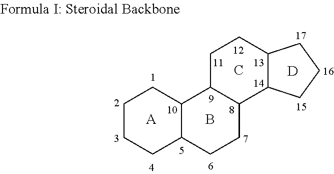

The two most commonly known classes of structural modifications of the cortisol steroid backbone to create glucocorticoid modulators include modifications of the 11-β hydroxy group and modification of the 17-β side chain (See, e.g., Lefebvre (1989) J. Steroid Biochem. 33:557-563).

As used herein, the term “mifepristone” refers to 11β-(4-dimethylaminophenyl)-17ß-hydroxy-17α-(1-propynyl)-estra-4,9-dien-3-one), also referred to as RU486, or as RU38.486, or as 17-beta-hydroxy-11-beta-(4-dimethyl-aminophenyl)-17-alpha-(1-propynyl)-estra-4,9-dien-3-one). Mifepristone binds to the glucocorticoid receptor (GR), typically with high affinity, and inhibits the biological effects initiated/mediated by the binding of any cortisol or cortisol analog to a GR receptor. Salts, hydrates and prodrugs of mifepristone are all included in the term “mifepristone” as used herein. Thus, used herein, “mifepristone” refers to the molecule that has the following structure:

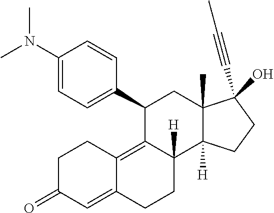

and to salts, hydrates and prodrugs thereof, and pharmaceutical compositions thereof.

As used herein, the phrase “non-steroidal backbone” in the context of glucocorticoid receptor modulators containing such refers to glucocorticoid receptor modulators that do not share structural homology to, or are not modifications of, cortisol. Such compounds include synthetic mimetics and analogs of proteins, including partially peptidic, pseudopeptidic and non-peptidic molecular entities.

Non-steroidal GRA compounds also include glucocorticoid receptor modulators having a cyclohexyl-pyrimidine backbone, a fused azadecalin backbone, a heteroaryl ketone fused azadecalin backbone, or an octahydro fused azadecalin backbone. Exemplary glucocorticoid receptor modulators having a cyclohexyl-pyrimidine backbone include those described in U.S. Pat. No. 8,685,973. Exemplary glucocorticoid receptor modulators having a fused azadecalin backbone include those described in U.S. Pat. Nos. 7,928,237; and 8,461,172. Exemplary glucocorticoid receptor modulators having an octohydro fused azadecalin backbone include those described in U.S. Provisional Patent Appl. No. 61/908,333, entitled Octahydro Fused Azadecalin Glucocorticoid Receptor Modulators, Attorney Docket No. 85178-887884 (007800US), filed on Nov. 25, 2013. Exemplary glucocorticoid receptor modulators and glucocorticoid receptor antagonists having a heteroaryl-ketone fused azadecalin backbone include those described in U.S. Pat. Nos. 8,859,774; 9,273,047; 9,707,223; and 9,956,216, all of which patents are hereby incorporated by reference in their entireties.

Exemplary heteroaryl-ketone fused azadecalin GRM compounds include, without limitation:

(R)-(1-(4-fluorophenyl)-6-((1-methyl-1H-pyrazol-4-yl) sulfonyl)-4,4a,5,6,7,8-hexahydro-1H-pyrazolo[3,4-g]isoquinolin-4a-yl) (4-(trifluoromethyl)pyridin-2-yl) methanone (termed “relacorilant”; also termed “CORT125134”), which has the following structure:

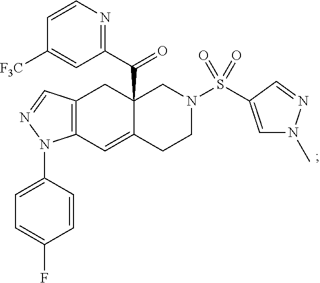 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

and
(R)-(1-(4-fluorophenyl)-6-((4-(trifluoromethyl)phenyl) sulfonyl)-4, 4a, 5,6,7,8-hexahydro-1-H-pyrazolo P,4-glisoquinolin-4a-yl) (pyridin-2-yl) methanone (termed “CORT113176”), which has the following structure:

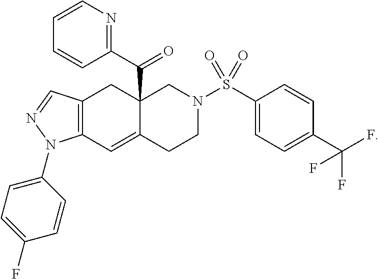 [SMILES: O=C(c1ccccn1)[C@]12Cc3cnn(-c4ccc(F)cc4)c3C=C1CCN(S(=O)(=O)c1ccc(C(F)(F)F)cc1)C2]

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, or physiological conditions.

Where substituent groups are specified by their conventional chemical formulae, written from left to right, they equally encompass the chemically identical substituents that would result from writing the structure from right to left, e.g., —CH₂O— is equivalent to —OCH₂—.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors and colors, and the like. One of skill in the art will recognize that other pharmaceutical excipients are useful in the present disclosure.

### I. DETAILED DESCRIPTIONS OF EMBODIMENTS

### A. Pituitary Tumor and SSTR Expression

Pituitary tumors cause excess adrenal cortisol secretion in patients, which results in a serious endocrine disorder known as Cushing's disease (CD). Somatostatin (SST) or somatostatin analogs and somatostain receptors (SSTRs) regulates growth hormone production and can be used for treatment of CD.

Somatostatin (SST) is a cyclincal peptide of 14 or 28 amino acids, known as SST14 and SST28, with a short half-life (<3 minutes). Cells that produce SST can be found in the central nervous system, peripheral nervous system, and in the endocrine pancreas and gut. In the central nervous system, after SST is released by the hypothalamus, it inhibit growth hormone secretion by interacting with one or more of the SSTRs.

Somatostain receptors, SSTR1, SSTR2, SSTR3, SSTR4, and SSTR5, are G-protein-coupled seven transmembrane receptors. Adult human pituitary glands mainly express SSTR1, SSTR2, SSTR3, and SSTR5. In normal human pituitary tissues, SSTRs are expressed in different abundance: SSTR5 is highly expressed, followed by SSTR2, SSTR1, SSTR3, and SSTR4. SSTR4 is expressed in extremely low levels in normal human pituitary tissues. SSTR2 and SSTR5 are prevalent and are expressed in 95% and 85% of growth hormone-secreting pituitary tumors, respectively. SSTR1 and SSTR3 each is expressed in about 40% of the pituitary tumors. See, Daniel Cuevas-Ramos and Maria Fleseriu, Drug. Des. Devel. Ther. 2016; 10:227-239.

In addition to somatostatin, somatostatin analogs can bind somatostatin receptors and regulate growth hormone secretion. Some of the somatostatin analogs have longer half-life and may have tighter binding activity than somatostatin. Some somatostatin analogs can bind to multiple types of SSTRs. The affinity of somatostatin analogs to different SSTRs may vary. In some cases, the somatostatin ligand preferentially binds or activates somatostatin receptor type 2 (SSTR2), for example, octreotide and lanreotide. In some cases, the somatostatin receptor ligand preferentially binds or activates somatostatin receptor type 5 (SSTR5), for example, pasireotide. In some cases, the somatostatin receptor ligand preferentially binds or activates SSTR2 and SSTR5. In some cases, the somatostatin receptor ligand preferentially binds or activates SSTR2, SSTR3, and SSTR5.

Drugs that target the somatostatin receptor (SSTR), expressed on the surface of corticotroph PitNETs, have been developed to treat CD [19]. For example, in two large prospective studies, pasireotide monotherapy normalized urinary free cortisol in up to 42% of patients with CD [21, 22]. Combination therapy may show promise whereby pasireotide administered together [23] with cabergoline (a dopamine receptor subtype 2 antagonist) and low-dose ketoconazole (a Hedgehog receptor smoothened (SMO) inhibitor that also inhibits cortisol synthesis) induced biochemical remission in six of eight patients at day 80, increasing the number of patients with a complete response to 88% [21]. However, to date, little was known about the receptor dynamics that may change during treatment of these drugs.

### B. Selective Glucocorticoid Receptor Modulator (SGRM)

Provided herein are SGRMs that can selectively stimulate SSTR5 expression in a patient having a pituitary tumor. In some embodiments, the increase in SSTR5 expression in the patient resulted from treatment with the SGRM is at least 20%, or 50%, or 100% than an increase in expression of another SSTR (for example, SSTR2) detected under the same assay conditions. In some instances, the increase in expression in SSTR5 can be at least 1.5-fold, at least 2-fold, at least 5-fold or even 10-fold higher than an increase in expression of SSTR2.

In some embodiments, the SGRM has a heteroaryl-ketone fused azadecalin backbone (“heteroaryl-ketone fused azadecalin SGRM”). In some embodiments, the heteroaryl-ketone fused azadecalin SGRM is selected from the group consisting of (i) relacorilant, also termed “CORT125134,”-and (ii) “CORT113176”), as disclosed above.

### C. Selective Stimulation of SSTRs

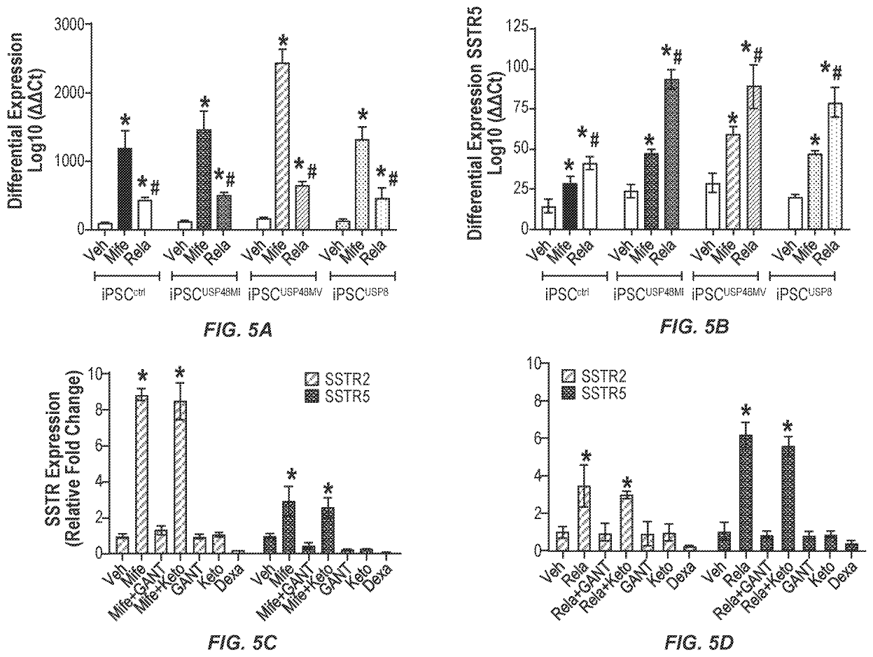

Applicant disclosed herein an in vitro system, i.e., the human PitNET Tissue organoids as a model for pituitary tumors (see Example 1) and that pituitary tumor cells respond differently to treatment of different types of SGRMs. For example, administration of a heteroaryl-ketone fused azadecalin SGRM to a patient having a pituitary tumor selectively increases the expression of SSTR5. In one illustrative example as shown in FIG. 5, administering relacorilant (a heteroaryl-ketone fused azadecalin SGRM) resulted in a greater induction of SSTR5 expression as compared to SSTR2. In contrast, mifepristone (a SGRM having a steroidal backbone and is not a heteroaryl-ketone fused azadecalin SGRM) resulted in a significantly greater induction in SSTR2 than SSTR5. Thus, the disclosure provides a method of selectively increasing expression of SSTR5 in a pituitary tumor by administering a SGRM. In some embodiments the SGRM that can selectively increase the expression of SSTR5 is a heteroaryl-ketone fused azadecalin glucocorticoid receptor modulator, for example, relacorilant.

The differential induction of the SSTRs as a resulted of administration of a SGRM disclosed herein can be confirmed by detecting the expression levels of SSTRs using methods well known in the art, for example, Western blots, flow cytometry, Enzyme-Linked Immunosorbent Assay (ELISA), quantitative PCR, and the like. Exemplary detection methods are described in Example 1.

### D. Induction of Apoptosis in Pituitary Tumor Tissues

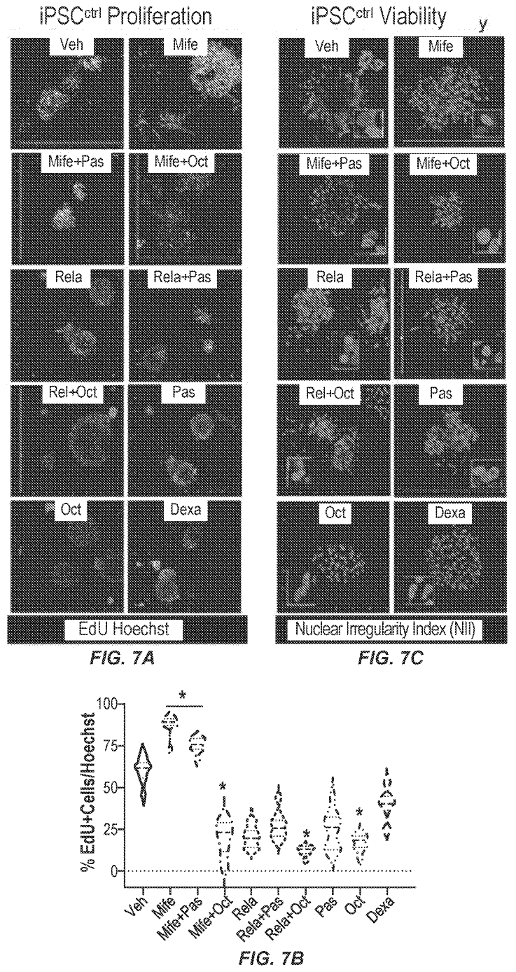

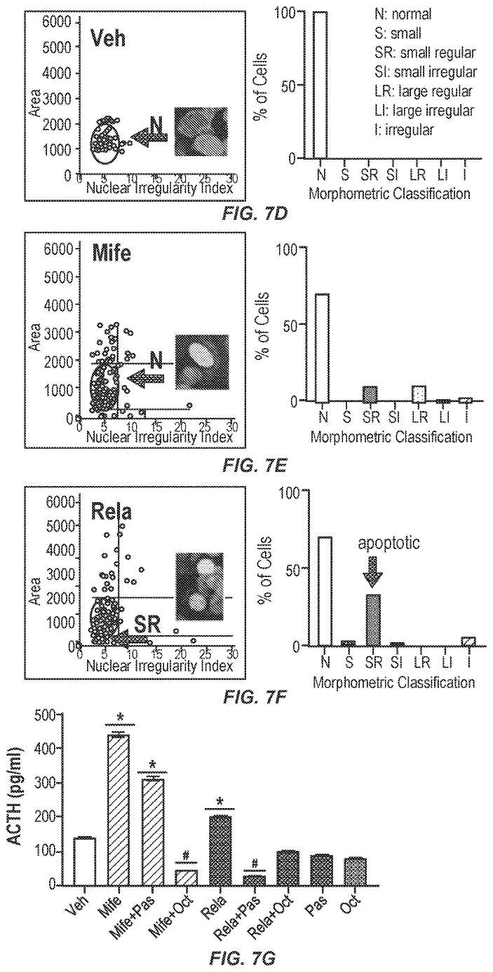

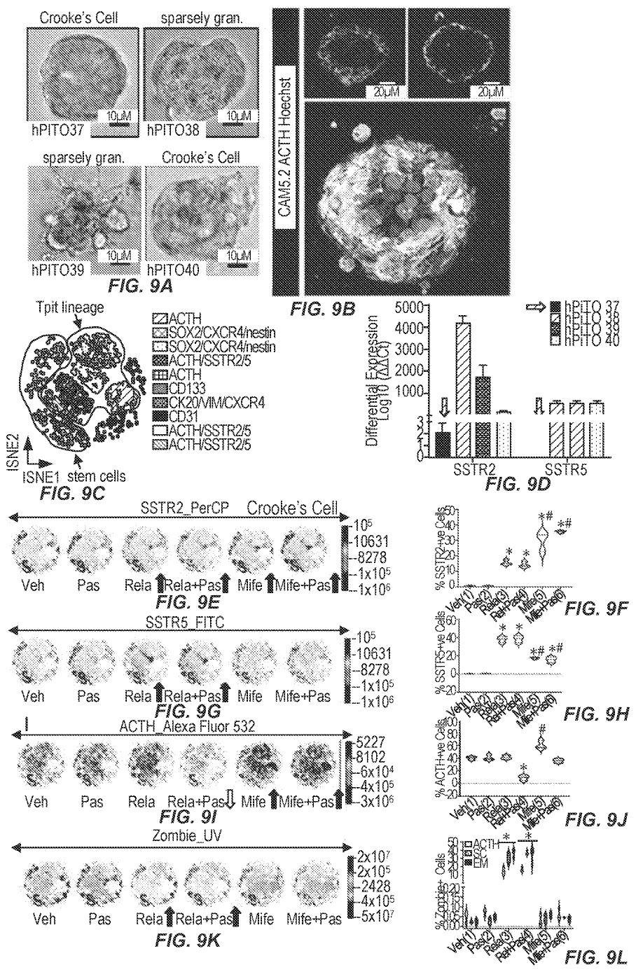

A heteroaryl-ketone fused azadecalin SGRM disclosed herein can be used to induce apoptosis in pituitary tumor cells and treat a pituitary tumor. In one illustrative example, a pituitary tumor organoid iPSC^{USP48M} treated by relacorilant, a heteroaryl-ketone fused azadecalin SGRM, showed an expression pattern of nuclear morphology consistent with increased apoptosis cells as compared to a control organoid. In contrast, mifepristone, a non-heteroaryl-ketone fused azadecalin SGRM, induces apoptosis in neither the tumor organoid iPSC^{USP48MV} nor the control organoid iPSC^{ctrl}. See FIG. 7C-F. In another illustrative example, the tumor organoid treated with relacorilant showed significant increase in cell death. See FIGS. 9K and 9L. Accordingly, disclosed herein is a method for inducing apoptosis in pituitary tumor cells and/or treating a pituitary tumor and the method comprises administering to the patient a SGRM, which is capable of selectively stimulating the expression of SSTR5. In some embodiments, the SGRM is a heteroaryl-ketone fused azadecalin SGRM, for example, relacorilant.

Apoptosis can be detected using methods known in the art, for example, methods that detect changes related to membrane alterations, DNA fragmentation, cytotoxicity and cell proliferation, mitochondrial damage. See, Gaspar Banfalvi, Apoptosis, 2017 February: 22 (2): 306-323; doi: 10.1007/s10495-016-1333-3, the entire disclosure of which is incorporated herein by reference.

In some cases, apoptosis of the pituitary tumor cells can be detected using a nuclear morphometric analysis (NMA). NMA includes nuclear image analysis and evaluation of size and regularity of adhered cells in culture. The method measures a large number of parameters related to nuclear morphology and performs principal component analysis to select the measurements that best separate regular from irregular nuclei. These measurements, including, for example, aspect, area box, radius ratio and roundness were combined into a single nuclear irregularity index (NII). Normal nuclei are used to set the parameters for a given cell type, and different nuclear phenotypes are separated in an area versus NII plot. This method provides a direct and objective way of screening normal, senescent, apoptotic and nuclear irregularities. Filippi-Chiela et al., PLOS ONE, Aug. 8, 2012, https://doi.org/10.1371/journal.pone.0042522. One exemplary method is described in Example 1, section 1.5.

Other approaches of detections of apoptosis can also be used in the methods disclosed herein include but are not limited to light-scattering flow cytometry and time-lapse microscopy.

### E. No Significant Increases in the Level of ACTH

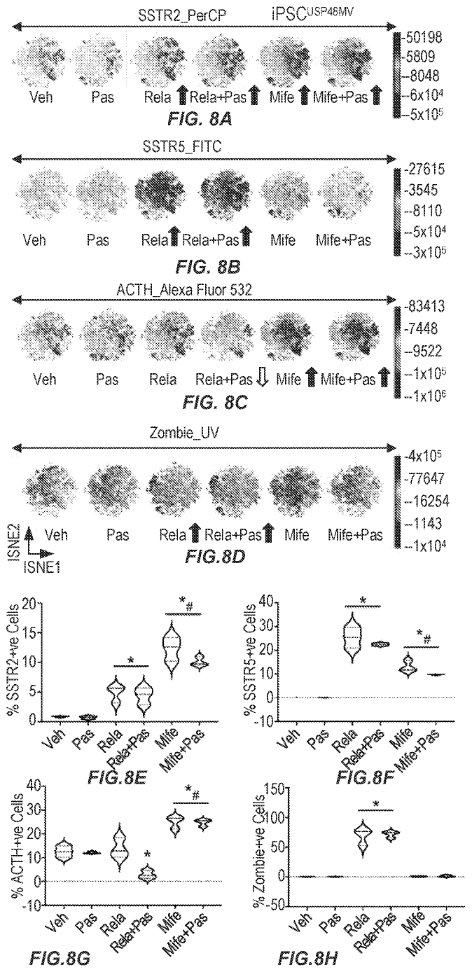

Advantageously, treating a patient having a pituitary tumor with a heteroaryl-ketone fused azadecalin SGRM (e.g., relacorilant) will not cause any significant increases in ACTH levels nor cause any significant increase in cortisol levels. Thus, this method of treatment can minimize the side effects commonly associated with excess cortisol, for example, risk of stroke, diabetes, obesity, depression, anxiety, etc. One illustrative example is shown in FIGS. 8C and 8G, which demonstrates that the administration of relacorilant did not significantly increase the number of ACTH-positive cells (i.e., cells expressing ACTH). In contrast, mifepristone, a non-heteroaryl-ketone fused azadecalin GRM, significantly increased the ACTH expression levels. Thus, as illustrated in FIG. 8G, unlike the pasireotide or relacorilant alone, and unlike the combination of mifepristone+pasireotide, the combination of relacorilant+pasireotide was uniquely capable of suppressing ACTH.

Furthermore, administration of a combination of a heteroaryl-ketone fused azadecalin GRM (e.g., relacorilant) and a somatostatin analog (e.g., pasireotide), can reduce ACTH expression levels in pituitary tissues. One illustrative example is shown in FIG. 8C.

Thus, the disclosure provides a method of selectively increasing expression of SSTR5, inducing apoptosis in pituitary tumor cells, and/or treating a pituitary tumor in a patient without resulting in significant increases in ACTH levels in the patient, and the method comprises administering a glucocorticoid receptor modulator (SGRM) to the patient, wherein the SGRM is capable of selectively stimulating the expression of SSTR5. In some embodiments, the SGRM is a heteroaryl-ketone fused azadecalin SGRM (e.g., relacorilant). Optionally, a somatostatin or a somatostatin analog is also administered to the patient having the pituitary tumor to reduce the ACTH levels.

Detection of ACTH can be performed using methods well known in the art, for example, Western blots, flow cytometry, ELISA, quantitative PCR, and the like. One exemplary method is described in Example 1, section 1.8.

### F. GRMS Modulates SSTR Expression Via the Hedgehog Pathway

Also disclosed herein is a method of activating the Hedgehog pathway signaling to modulate SSTR expression. Disclosed herein is a method of modulating SSTR expression (e.g., selectively stimulating SSTR5 expression) through a Hedgehog signaling pathway, and the method comprises administering to a patient a heteroaryl-ketone fused azadecalin GRM disclosed herein (e.g., relacorilant). In some embodiments, the Hedgehog pathway is a non-canonical Hedgehog signaling pathway. In some embodiments, the non-canonical Hedgehog signaling pathway activates GLI independent of SMO.

The Hedgehog (HH) pathways include canonical Hedgehog signaling pathways and non-canonical Hedgehog signaling pathways, both involved in carcinogenesis. A canonical HH signaling pathway is activated by binding of HH ligands, e.g., Sonic Hedgehog (SHH), Indian hedgehog homolog (IHH), and Desert hedgehog (DHH), to their receptors, patched-1 (PTCH) on the surface of target cells. The HH ligand-bound PTCH lost its inhibitory regulation on Smoothened (SMO), resulting in activation of SMO, which in turn activates downstream glioma-associated homolog (GLI) transcription factors, GLI1 and GLI2. The activated GLI1 and GLI2 in turn regulates expression of target genes that are involved in cell proliferation survival, self-renewal, and invasiveness.

In general, non-canonical HH signaling pathways include 1) Type I pathways involving signaling through Protein patched homolog 1 (PTCH1) independent of SMO, 2) Type II pathways involving signaling through steroid monooxygenase (SMO) independent of GLI, and 3) SMO-independent activation of GLI such as that observed in the current study [42, 43]. In SMO-independent activation of GLI pathway, GLI1 and/or GLI2 activate proteins in the oncogenic intracellular signaling pathways such as PI3-AKT, TGFβ-smad, and Ras-Raf-MEK-ERK, and this activation is independent of HH ligands. See, Harada et al., Int. J. Mol. Sci. 2020 May; 21 (9) 3126, doi: 10.3390/ijms21093126; and Teperino et al., Semin Cell Dev. Biol. 2014 September; 0:81-92, the entire content of each publication is incorporated herein by reference.

An HH signal pathway can be inhibited by agents that interact with players (e.g., SMO, GLI1, and GLI2) in the pathway and block their activation. For example, the SMO inhibitor ketoconazole and the GLI1 and GLI2 inhibitor GANT61 reduce cell proliferation and cell viability by blocking activation of their respective targets.

Surprisingly, Applicant discovered that a SGRM disclosed herein activates SSTR through a Hedgehog signaling pathway. In some embodiments, a SGRM stimulates the expression of an SSTR via an SMO-independent non-canonical HH signaling pathway. Results from one exemplary study (shown in FIG. 5) show that ketoconazole (an SMO inhibitor) had no effect on the induction of SSTR expression while GANT61 (an GLI inhibitor) reduced the induction of SSTR5 expression by relacorilant. Results from another exemplary study (also shown in FIG. 5) show that ketoconazole had no effect on the induction of SSTR2 expression while GANT61 reduced the induction of SSTR2 expression by mifepristone. Accordingly, this disclosure provides a method of activating a Hedgehog pathway to modulate SSTR expression or modulate SSTR expression through a Hedgehog signaling pathway (e.g., an SMO-independent non-canonical Hh signaling pathway) in a patient, and the method comprises administering to the patient a SGRM. In some embodiments the SSTR is SSTR2 and the SGRM is mifepristone. In some embodiments, the SSTR is SSTR5 and the SGRM is a heteroaryl-ketone fused azadecalin SGRM, such as relacorilant.

The activation of an HH signaling pathway associated with the treatment of a GRM disclosed herein can be detected by detecting the expression and activation of the target genes in the pathway, for example, detecting the expression of GLI1 and GLI2 and/or activation of the PI3-AKT, TGFβ-smad, and Ras-Raf-MEK-ERK, and the like.

G. Diagnosing Pituitary Tumor with a GRM Selectively Stimulating SSTR5

Administration of a heteroaryl-ketone fused azadecalin GRM, such as relacorilant, results in an increase in SSTR5 expression on pituitary tumor cells, and such an increase allows ease of tumor detection in a patient by using agents that specifically recognizes SSTR5. In one embodiment, the method comprises administering a heteroaryl-ketone fused azadecalin GRM to the patient suspected of having a pituitary tumor, detecting expression of SSTR5, using an agent that specifically binds SSTR5, thereby detecting the pituitary tumor. In some embodiments, the SSTR5 expression level detected after the administration of the heteroaryl-ketone fused azadecalin GRM is increased as compared to (i) the SSTR5 level before the treatment of the heteroaryl-ketone fused azadecalin GRM, or (ii) the SSTR5 level in the normal issue of the patient. In some embodiments, the agent that specifically binds SSTR5 is a somatostatin or a somatostatin analog that is conjugated to a detectable label, for example, a label that can produce fluorescent or luminescent signals, which can be detected and quantified. A label suitable for use with the methods disclosed herein can be a fluorescent label, a chromogenic label, or an electrochemical label. Exemplary fluorescent labels for use with the methods disclosed herein include water-soluble rhodamine dyes, fluoresceins, 4,7-dichlorofluoresceins, benzoxanthene dyes and energy transfer dyes, and the like. Exemplary labels that can be used are also disclosed in e.g., Handbook of Molecular Probes and Research Reagents, 8th ed., 2002, Molecular Probes, Eugene, OR; U.S. Pat. Nos. 4,318,846, 4,997,928, 5,945,526, 6,096,723, 6,191,278, and 6,372,907, and Lee et al., 1997, Nucleic Acids Research 25:2816-2822. The entire content of said publications and patents are incorporated herein by reference.

### H. Pharmaceutical Compositions of Glucocorticoid Receptor Modulators

The composition comprising a SGRM of the present disclosure can be prepared in a wide variety of oral, parenteral and topical dosage forms. Oral preparations of either include tablets, pills, powder, dragees, capsules, liquids, lozenges, cachets, gels, syrups, slurries, suspensions, etc., suitable for ingestion by the patient. A SGRM composition of the present disclosure can also be administered by injection, that is, intravenously, intramuscularly, intracutaneously, subcutaneously, intraduodenally, or intraperitoneally. Also, a SGRM composition described herein can be administered by inhalation, for example, intranasally. Additionally, a SGRM composition of the present disclosure can be administered transdermally. A SGRM composition of this disclosure can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Accordingly, the present disclosure provides pharmaceutical compositions of a SGRM including a pharmaceutically acceptable carrier or excipient and a SGRM compound of the present disclosure.

For preparing pharmaceutical compositions from a SGRM of the present disclosure, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% or 10% to 70% of the compounds of the present disclosure.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the disclosure can also be used orally using, for example, push-fit capsules made of gelatin, as well as soft, sealed capsules made of gelatin and a coating such as glycerol or sorbitol. Push-fit capsules can contain the compounds of the present disclosure mixed with a filler or binders such as lactose or starches, lubricants such as talc or magnesium stearate, and, optionally, stabilizers. In soft capsules, the compounds of the present disclosure may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the compounds of the present disclosure are dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving one or more compounds of the present disclosure in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compounds of the present disclosure in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the disclosure can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The SGRM compositions provided herein can also be delivered as microspheres for slow release in the body. For example, microspheres can be formulated for administration via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In another embodiment, the SGRM compositions of the present disclosure can be formulated for parenteral administration, such as intravenous (IV) administration or administration into a body cavity or lumen of an organ. The formulations for administration will commonly comprise a solution of the compositions of the present disclosure dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose, any bland fixed oil can be employed including synthetic mono- or diglycerides. In addition, fatty acids such as oleic acid can likewise be used in the preparation of injectables. These solutions are sterile and generally free of undesirable matter. These SGRM formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present disclosure in these formulations can vary widely, and will be selected primarily based on fluid volumes, viscosities, body weight, and the like, in accordance with the particular mode of administration selected and the patient's needs. For IV administration, the SGRM formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the compositions of the present disclosure can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present disclosure into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

Lipid-based drug delivery systems include lipid solutions, lipid emulsions, lipid dispersions, self-emulsifying drug delivery systems (SEDDS) and self-microemulsifying drug delivery systems (SMEDDS). In particular, SEDDS and SMEDDS are isotropic mixtures of lipids, surfactants and co-surfactants that can disperse spontaneously in aqueous media and form fine emulsions (SEDDS) or microemulsions (SMEDDS). Lipids useful in the formulations of the present disclosure include any natural or synthetic lipids including, but not limited to, sesame seed oil, olive oil, castor oil, peanut oil, fatty acid esters, glycerol esters, Labrafil®, Labrasol®, Cremophor®, Solutol®, Tween®, Capryol®, Capmul®, Captex®, and Peceol®

The SGRM composition can also contain other compatible therapeutic agents. In some embodiments, the other therapeutic agents include a somatostatin or a somatostatin analog, for example, pasireotide. The compounds described herein can also be used in combination with one another, or with other active agents known to be useful in antagonizing a glucocorticoid receptor, or with adjunctive agents that may not be effective alone but may contribute to the efficacy of the active agent.

### I. Methods of Administration

In some embodiments, a method disclosed herein comprises administering a heteroaryl-ketone fused azadecalin SGRM to a patient having a pituitary tumor. In some embodiments, the method comprises administering a heteroaryl-ketone fused azadecalin SGRM and somatostatin or a somatostatin analog, for example, pasireotide. Any suitable dose of relacorilant or other heteroaryl-ketone fused azadecalin SGRM (e.g., CORT122928 or CORT113176) may be used in combination with somatostatin or a somatostatin analog in the methods disclosed herein.

A SGRM composition of the present disclosure can be delivered by any suitable means, including oral, parenteral (e.g., intravenous injection or intramuscular injection) and topical methods. Transdermal administration methods, by a topical route, can be formulated as applicator sticks, solutions, suspensions, emulsions, gels, creams, ointments, pastes, jellies, paints, powders, and aerosols.

The pharmaceutical preparation is preferably in unit dosage form. In such form the preparation is subdivided into unit doses containing appropriate quantities of the compounds and compositions of the present disclosure. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

SGRMs can be administered orally. For example, the SGRM can be administered as a pill, a capsule, or liquid formulation as described herein. Alternatively, SGRMs can be provided via parenteral administration. For example, the SGRM can be administered intravenously (e.g., by injection or infusion). Additional methods of administration of the compounds described herein, and pharmaceutical compositions or formulations thereof, are described herein.

In some embodiments, the SGRM is administered in one dose. In other embodiments, the SGRM is administered in more than one dose, e.g., 2 doses, 3 doses, 4 doses, 5 doses, 6 doses, 7 doses, or more. In some cases, the doses are of an equivalent amount. In other cases, the doses are of different amounts. The doses can increase or taper over the duration of administration. The amount will vary according to, for example, the SGRM properties. To determine an effective dose, the SGRM must elevate the level of cortisol to above 18 μg/dL in a normal, healthy adult with normal adrenal function.

### J. Exemplary Embodiments

This disclosure provides the following non-limiting embodiments.

Embodiment 1 is a method of selectively increasing SSTR5 expression in human pituitary cells in a patient, the method comprising administering to the patient a selective glucocorticoid receptor modulator (SGRM) to the patient, wherein the SGRM is capable of selectively stimulating SSTR5 expression.

Embodiment 2 is the method of Embodiment 1, wherein the method further comprises detecting an increase in SSTR5 expression.

Embodiment 3 is a method of inducing apoptosis in pituitary tumor cells or treating a pituitary tumor in a patient without resulting in significantly increasing the level of ACTH, the method comprising administering a SGRM to the patient, wherein the SGRM is capable of selectively stimulating SSTR5 expression.

Embodiment 4 is the method of any one of Embodiments 1-3, wherein the SGRM is a heteroaryl-ketone fused azadecalin SGRM.

Embodiment 5 is the method of Embodiment 4, wherein administering to the SGRM does not significantly increase the expression level of ACTH in the patient.

Embodiment 6 is the method of Embodiment 4, wherein the heteroaryl-ketone fused azadecalin SGRM is a (R)-(1-(4-fluorophenyl)-6-((1-methyl-1H-pyrazol-4-yl) sulfonyl)-4,4a,5,6, 7,8-hexahydro-1Hpyrazolo[-3,4-g]isoquinolin-4a-yl) (4-(trifluoromethyl)pyridin-2-yl) methanone (relacorilant) having the structure:

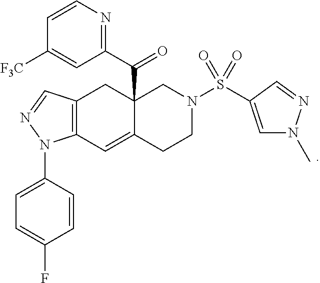 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

Embodiment 7 is the method of any one of Embodiments 1-3, wherein the method further comprises administering a somatostatin or somatostatin analog.

Embodiment 8 is the method of Embodiment 7, wherein the somatostatin analog selectively binds to SSTR5.

Embodiment 9 is the method of Embodiment 8, wherein the somatostatin analog is pasireotide.

Embodiment 10 is a method of detecting a pituitary tumor in a patient, the method comprising administering a SGRM to the patient, wherein the SGRM selectively stimulates the expression of SSTR5 in the pituitary tumor cells and administering an agent that selectively binds to the SSTR5, and the agent that bind to SSTR5.

Embodiment 11 is the method of Embodiment 10, wherein the pituitary tumor is a neuroendocrine tumor.

Embodiment 12 is the method of Embodiment 10, wherein an expression level of SSTR5 before administering the SGRM was undetectable, or only poorly detectable, with imaging. The expression level of SSTR5 is undetectable means that the SSTR5 level is lower than the detectable limit of an equipment used for the dection.

Embodiment 13 is the method of Embodiment 10, wherein the agent that selectively binds to SSTR5 is a labeled somatostatin or somatostatin analog.

Embodiment 14 is the method of Embodiment 10, wherein the SGRM is a heteroaryl-ketone fused azadecalin SGRM.

Embodiment 15 is the method of Embodiment 14, wherein the heteroaryl-ketone fused azadecalin SGRM is a (R)-(1-(4-fluorophenyl)-6-((1-methyl-1H-pyrazol-4-yl) sulfonyl)-4,4a,5,6, 7,8-hexahydro-1H-pyrazolo[-3,4-g]isoquinolin-4a-yl) (4-(trifluoromethyl)pyridin-2-yl) methanone (relacorilant) having the structure:

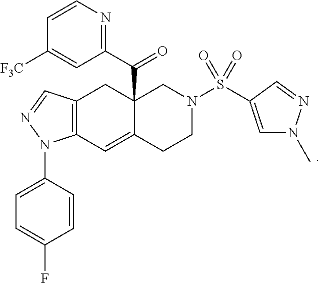 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

Embodiment 16 is the method of Embodiment 10, wherein the method further comprises imaging the patient, effective to provide an image of the tumor within the patient wherein the pituitary tumor is localized.

Embodiment 17. The method of any of Embodiments 10 to 16, wherein said detecting a pituitary tumor in a patient comprises detecting an increased expression of SSTR5 as compared to (i) the SSTR5 level before the treatment of the SGRM or (ii) the SSTR5 level in the normal tissue.

Embodiment 18. The method of any of Embodiments 10 to 17, wherein administering to the SGRM does not significantly increase ACTH expression in the patient.

Embodiment 19. The use of a selective glucocorticoid receptor modulator (SGRM) for the treatment of Cushing's syndrome by the selective increase of SSTR5 expression, wherein the SGRM is capable of selectively stimulating the expression of SSTR5.

Embodiment 20. The use of a selective glucocorticoid receptor modulator (SGRM) in the manufacture of a medicament for treating Cushing's syndrome by the selective increase of SSTR5 expression, wherein the SGRM is capable of selectively stimulating the expression of SSTR5.

Embodiment 21. The use of Embodiment 19 or Embodiment 20, wherein the SGRM s a heteroaryl-ketone fused azadecalin compound.

Embodiment 22. The use of Embodiment 19 or Embodiment 20, wherein the SGRM is the heteroaryl-ketone fused azadecalin compound (R)-(1-(4-fluorophenyl)-6-((1-methyl-1H-pyrazol-4-yl) sulfonyl)-4,4a,5,6,7,8-hexahydro-1H-pyrazolo[-3,4-g]isoquinolin-4a-yl) (4-(trifluoromethyl)pyridin-2-yl) methanone (relacorilant) having the structure:

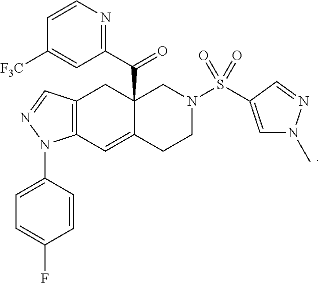 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

Embodiment 23. The use of any of Embodiments 19 to 22, wherein the use comprises use with a somatostatin or somatostatin analog which binds to SSTR5.

Embodiment 24. The use of any of Embodiments 19 to 22, wherein the use comprises use with the somatostatin analog pasireotide.

Embodiment 25. The use of a selective glucocorticoid receptor modulator (SGRM) for treating a pituitary tumor without significantly increasing the level of ACTH, by the selective increase of SSTR5 expression, wherein the SGRM is capable of selectively stimulating the expression of SSTR5.

Embodiment 26. The use of a selective glucocorticoid receptor modulator (SGRM) in the manufacture of a medicament for treating a pituitary tumor without significantly increasing the level of ACTH, by the selective increase of SSTR5 expression, wherein the SGRM is capable of selectively stimulating the expression of SSTR5.

Embodiment 27. The use of Embodiment 25 or of Embodiment 26, wherein the SGRM is a heteroaryl-ketone fused azadecalin compound.

Embodiment 28. The use of Embodiment 25 or of Embodiment 26, wherein the SGRM is the heteroaryl-ketone fused azadecalin compound (R)-(1-(4-fluorophenyl)-6-((1-methyl-1H-pyrazol-4-yl) sulfonyl)-4,4a,5,6,7,8-hexahydro-1H-pyrazolo[-3,4-g]isoquinolin-4a-yl) (4-(trifluoromethyl)pyridin-2-yl) methanone (relacorilant) having the structure:

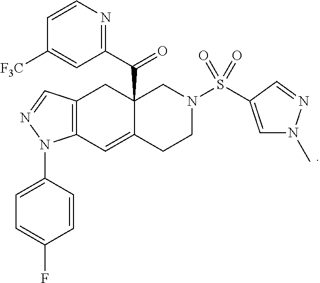 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

Embodiment 29. The use of any of Embodiments 25 to 28, wherein the use comprises use with a somatostatin or somatostatin analog which binds to SSTR5.

Embodiment 30. The use of any of Embodiments 25 to 28, wherein the use comprises use with the somatostatin analog pasireotide.

Embodiment 31. The use of a selective glucocorticoid receptor modulator (SGRM) for inducing apoptosis in a pituitary tumor without significantly increasing the level of ACTH, by the selective increase of SSTR5 expression, wherein the SGRM is capable of selectively stimulating the expression of SSTR5.

Embodiment 32. The use of a selective glucocorticoid receptor modulator (SGRM) in the manufacture of a medicament for inducing apoptosis in a pituitary tumor without significantly increasing the level of ACTH, by the selective increase of SSTR5 expression, wherein the SGRM is capable of selectively stimulating the expression of SSTR5.

Embodiment 33. The use of Embodiment 31 or of Embodiment 32, wherein the SGRM is a heteroaryl-ketone fused azadecalin compound.

Embodiment 34. The use of Embodiment 31 or of Embodiment 32, wherein the SGRM is the heteroaryl-ketone fused azadecalin compound (R)-(1-(4-fluorophenyl)-6-((1-methyl-1H-pyrazol-4-yl) sulfonyl)-4,4a,5,6,7,8-hexahydro-1H-pyrazolo[-3,4-g]isoquinolin-4a-yl) (4-(trifluoromethyl)pyridin-2-yl) methanone (relacorilant) having the structure:

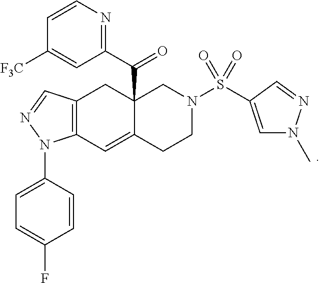 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

Embodiment 35. The use of any of Embodiments 31 to 34, wherein the use comprises use with a somatostatin or somatostatin analog which binds to SSTR5.

Embodiment 36. The use of any of Embodiments 31 to 34, wherein the use comprises use with the somatostatin analog pasireotide.

Embodiment 37. The use of a selective glucocorticoid receptor modulator (SGRM) for detecting a pituitary tumor in a patient, wherein the SGRM selectively stimulates the expression of SSTR5 in the pituitary tumor cells, wherein said use comprises use with an agent that selectively binds to the SSTR5.

Embodiment 38. The use of Embodiment 37, wherein the pituitary tumor is a neuroendocrine tumor.

Embodiment 39. The use of Embodiment 37, wherein an expression level of SSTR5 before administering the SGRM was undetectable, or only poorly detectable, with imaging.

Embodiment 40. The use of Embodiment 37, wherein the agent is a labeled somatostatin or somatostatin analog.

Embodiment 41. The use of Embodiment 37, wherein the SGRM is a heteroaryl-ketone fused azadecalin SGRM.

Embodiment 42. The use of Embodiment 41, wherein the heteroaryl-ketone fused azadecalin SGRM is (R)-(1-(4-fluorophenyl)-6-((1-methyl-1H-pyrazol-4-yl) sulfonyl)-4,4a,5,6, 7,8-hexahydro-1H-pyrazolo[-3,4-g]isoquinolin-4a-yl) (4-(trifluoromethyl)pyridin-2-yl) methanone (relacorilant) having the structure:

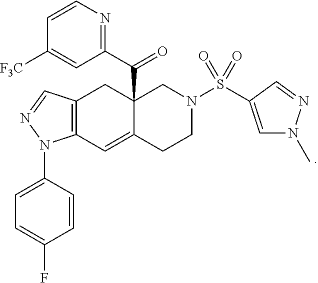 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

Embodiment 43. The use of a selective glucocorticoid receptor modulator (SGRM), wherein the SGRM is capable of selectively stimulating the expression of SSTR5, for detecting a pituitary tumor in a patient.

Embodiment 44. The use of Embodiment 43, further comprising use with a somatostatin or somatostatin analog.

Embodiment 45. The use of Embodiment 43, wherein the pituitary tumor is a neuroendocrine tumor.

Embodiment 46. The use of Embodiment 43, wherein the somatostatin or somatostatin analog is a labeled somatostatin or somatostatin analog.

Embodiment 47. The use of Embodiment 43, wherein the SGRM is a heteroaryl-ketone fused azadecalin SGRM.

Embodiment 48. The use of Embodiment 47, wherein the heteroaryl-ketone fused azadecalin SGRM is (R)-(1-(4-fluorophenyl)-6-((1-methyl-1H-pyrazol-4-yl) sulfonyl)-4,4a,5,6, 7,8-hexahydro-1H-pyrazolo[-3,4-g]isoquinolin-4a-yl) (4-(trifluoromethyl)pyridin-2-yl) methanone (relacorilant) having the structure:

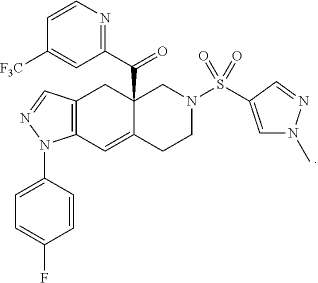 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

Embodiment 49. The use of Embodiment 43, wherein the use further comprises use with an image of a tumor.

Embodiment 50. The use of Embodiment 37, wherein the use further comprises imaging the patient, effective to provide an image of the tumor within the patient wherein the pituitary tumor is localized, without resulting in significant increases in adrenocorticotropic hormone (ACTH) in the patient.

Although the foregoing embodiments has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

### III. EXAMPLES

### 1. Materials and Methods

### 1.1 Generation, Culture and Differentiation of Induced Pluripotent Stem Cells (IPSCs) to Develop PitNET Organoids

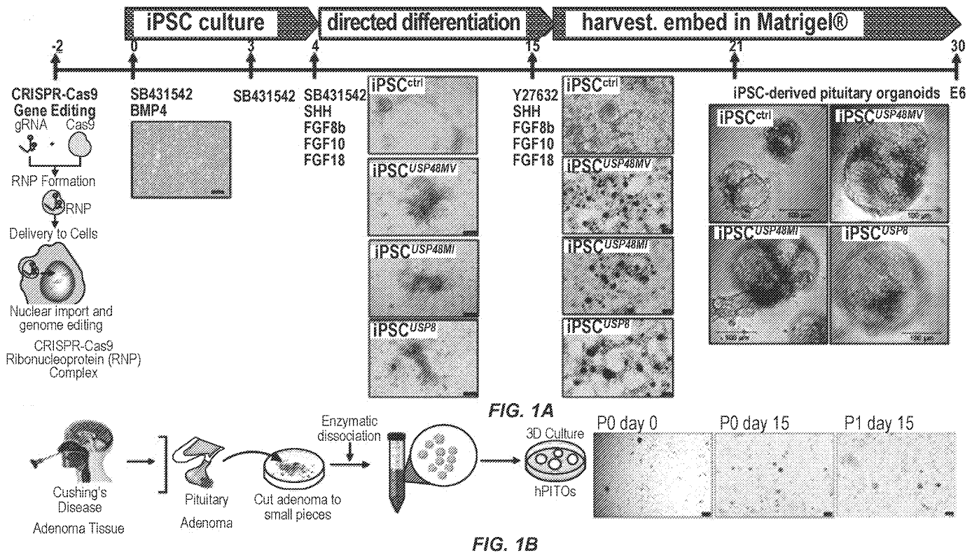

Peripheral Blood Mononuclear Cells (PBMCs) from a healthy individual (JCAZ001) were used to generate iPSCs. Six well culture plates coated with 2 mL/well 0.67% Matrigel™ (diluted in E8 media, UA iPSC core, 151169-01) were used to initially culture and expand the iPSCs. The iPSC lines were reprogrammed at the University of Arizona iPSC Core using an established protocol [24]. Once reaching 70% confluency, iPSCs were passaged to Matrigel™ coated 24 well plates at a ratio of 1:8 and grown to 85-90% confluency before beginning the directed differentiation schedule (FIG. 1A) that followed the culture of cells as follows: 1) 0 to 3 with E6 media supplemented with 1% penicillin/streptomycin, 10 μM SB431542 and 5 ng/ml Bone Morphogenetic Protein 4 (BMP4), 2) at day 3 BMP4 was withdrawn, 3) at day 4 cells were cultured in E6 media, supplemented with 10 μM SB431542, 30 ng/ml human recombinant SHH, 100 ng/ml FGF8b, 10 ng/ml FGF18 and 50 ng/ml FGF10, and 4) cells were harvested at day 15 using cold E6 media and resuspended in Matrigel™ (20,000 cells/50 μl Matrigel™). Matrigel™ domes containing iPSCs were plated in culture dishes and overlaid with differentiation media (E6 media supplemented with 10 μM Y-27632, 30 ng/ml human recombinant SHH, 100 ng/ml FGF8b, 10 ng/ml FGF18 and 50 ng/ml FGF10, Table 2), and cultured for a further 15 days at 37° C. at a relative humidity of 95% and 5% CO₂ during which time the 3D organoids developed and used for experiments and analyses. All human iPSC lines were negative for mycoplasma contamination as tested using the Mycoalert Mycoplasma testing kits (LT07-318, Lonza) and no karyotype abnormalities were found (KaryoStat+, Thermo).

TABLE 1
Clinical Characteristics of Pituitary Adenoma Samples Used for the Generation of
Organoids

| Organoid | | | | Clinical | Prior |
|---|---|---|---|---|---|
| Line | Gender | Age | Neuropathology Report | Diagnosis | Treatment |
| hPITO37 | F | 53 | Crooke cell adenoma. T-PIT+, | Cushing's | None |
| | | | ACTH+, CAM5.2+, | disease | |
| | | | CK20+, synaptophysin+, MIB (Ki67) | | |
| | | | LI: <1% | | |
| hPITO38 | F | 43 | Corticotroph subtype, sparsely. T- | Cushing's | None |
| | | | PIT+, ACTH+, CAM5.2+, CK20+, | disease | |
| | | | synaptophysin+, MIB (Ki67) LI: | | |
| | | | <1% | | |
| hPITO39 | M | 62 | Corticotroph subtype, sparsely | Cushing's | None |
| | | | granulated, T-PIT+, ACTH+ | disease | |
| | | | CAM5.2+, CK20+, synaptophysin+, | | |
| | | | MIB | | |
| | | | (Ki67) LI: <1% | | |
| hPITO40 | F | 28 | Corticotroph subtype, Crooke cell | Cushing's | None |
| | | | adenoma , T- | disease | |
| | | | PIT+, ACTH+, CAM5.2+, CK20+, | | |
| | | | synaptophysin+, alpha subunit+, MIB | | |
| | | | (Ki67) LI: 2.5% | | |

TABLE 2
Composition of iPSC Differentiation and Organoid Growth Media

| Media | | | Stock | | Active | |
|---|---|---|---|---|---|---|
| Name | Component | Catalog Number | Conc. | | Conc. | |
| Day | E8 Media | UA iPSC core # 151169-01 | 100% | | — | |
| 0-2 | Penicillin/ | Thermo Fisher Scientific # | 100X | | 1X | |
| | Streptomycin | 15140122 | | | | |
| | Y27632 | SIGMA # Y0503 | 10 | mM | 10 | μM |
| Day 3 | E6 | Thermo Fisher Scientific # | 100% | | — | |
| | | A1516401 | | | | |
| | BMP4 | R&D Systems # 314-BP | 5 | mg/mL | 5 | ng/mL |
| | SB431542 | R&D Systems # 1614/10 | 10 | mM | 10 | μM |
| | Penicillin/ | Thermo Fisher Scientific # | 100X | | 1X | |
| | Streptomycin | 15140122 | | | | |
| Day | E6 | Thermo Fisher Scientific # | 100% | | — | |
| | | A1516401 | | | | |
| 4-14 | Penicillin/ | Thermo Fisher Scientific # | 100X | | 1X | |
| | Streptomycin | 15140122 | | | | |
| | SB431542 | R&D Systems # 1614/10 | 10 | mM | 10 | μM |
| | hSHH | R&D Systems # 8908-SH | 200 | μg/mL | 30 | ng/mL |
| | FGF10 | Peprotech # 100-26 | 100 | μg/mL | 50 | ng/mL |
| | FGF8 | R&D Systems # 423-F8 | 25 | μg/mL | 100 | ng/mL |
| | FGF18 | R&D Systems # 8988-F18 | 10 | ng/mL | 50 | μg/mL |
| Day | E6 | Thermo Fisher Scientific # | 100% | | — | |
| | | A1516401 | | | | |
| 15-30 | Penicillin/ | Thermo Fisher Scientific # | 100X | | 1X | |
| | Streptomycin | 15140122 | | | | |
| | Y27632 | SIGMA # Y0503 | 10 | mM | 10 | μM |
| | hSHH | R&D Systems # 8908-SH | 200 | μg/mL | 30 | ng/mL |
| | FGF10 | Peprotech # 100-26 | 100 | μg/mL | 50 | ng/mL |
| | FGF8 | R&D Systems # 423-F8 | 25 | μg/mL | 100 | ng/mL |
| | FGF18 | R&D Systems # 8988-F18 | 10 | ng/mL | 50 | μg/mL |

1.2 CRISPR/Cas9 Gene Editing and Validation of iPSCs

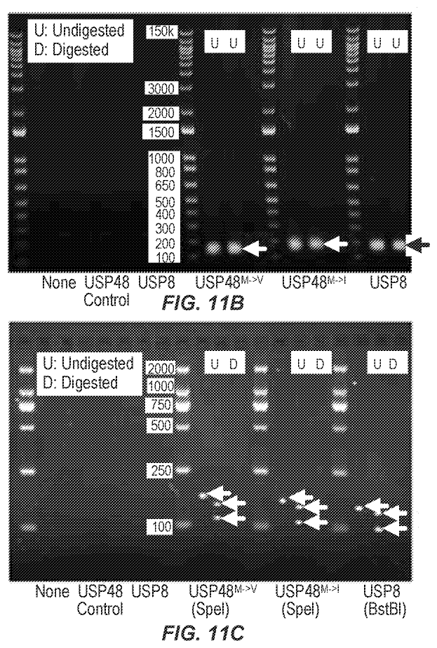

A CRISPR/Cas9 approach was used to genetically engineer iPSCs to express known somatic USP48 and USP8 mutations of CD [25-28]. Mutation sgRNAs for either USP8 (Gene ID: 9101, Double: Y717C (A2150G) & S718P (T2152C)), and USP48 (Gene ID: 84196; M415I (G1245A) and M415V (A1243G), FIG. 11A) were created using the CRISPR Design Tool (SIGMA VC40007) with the assistance of the SIGMA Bioinformatics Support Team. Single guide RNAs (sgRNAs) targeted specific restriction sites whereby USP8 destroyed a BseRI site and created a BstBI site, and USP48 created a SpeI site. SgRNAs were cloned into a LV01/pLV-U6g-EPCG expression vector (expressing sgRNA and Cas9), and combined with Cas9 protein (SIGMA E120030), warm Opti-MEM™ I Reduced Serum Medium (Thermo Fisher Scientific 31985-070) and TransIT-CRISPR (SIGMA T1706). These TransIT-CRISPR Cas9 Ribonucleotide complexes were then added dropwise to their respective iPSC plates and incubated for 48 hours before starting the initiation of the differentiation schedule.

Successful gene editing of the iPSCs was validated using restriction fragment length polymorphism (RFLP) analysis. DNA was extracted from iPSCs using the QIAamp DNA Micro Kit (Qiagen 56304) according to manufacturer's protocol, and the concentration was measured using a NanoDrop™ One/OneC Microvolume UV-Vis Spectrophotometer (Thermo Fisher Scientific ND-ONE-W). Amplification of 300 ng of DNA was performed using KOD Hot Start Master Mix (Sigma-Aldrich 71842), forward and reverse primers of the mutations (USP8 Forward: TTG AAG TTT ATC GCC ATT TTA TTC G (SEQ ID NO:10), Reverse: GCT GGT ATA GCC ATC CAC AGA A (SEQ ID NO:11); USP48 Forward: TGT TGT TCT AGG TTT GTT CCC CA (SEQ ID NO:12), Reverse: TCA CCA ACA TTC ACC TTA TGG AA (SEQ ID NO:13). The samples were amplified in a thermal cycler (Applied Biosystems Veriti™ 96-Well Thermal Cycler, Thermo Fisher Scientific 4375786) with the following temperature cycles: 95° for 2 minutes, 40 cycles of 95° C. for 20 seconds, 48° C. for 10 seconds, 70° C. for 1 minute. 6× Purple Gel Loading Dye (NEB B7025S) was added to the amplicon and the PCR products were loaded onto a 1% agarose gel. After electrophoresis, the bands were visualized using a UV imager, extracted using the GenElute™ Gel Extraction Kit (SIGMA NA1111) and purified with the GenElute™ PCR Clean-Up Kit (SIGMA NA1020). 300 ng of the eluted DNA was digested overnight at 37° C. with the restriction enzymes including USP8-BstBI and USP48-SpeI. Restriction fragments were visualized on a 2.5% agarose gel alongside undigested DNA (FIG. 11B, C).

1.3 Generation and Culture of Human PitNET Tissue Organoids (hPITOs)

Patients undergoing planned transsphenoidal surgery for PitNETs were identified in the outpatient neuroendocrinology and neurosurgery clinics and collected by the St. Joseph's Hospital and Barrow Neurological Institute Biobank under the collection protocol PHXA-05TS038, and outcomes data protocol PHXA-0004-72-29 with approval of the Institutional Review Board (IRB) and patient consent. De-identified samples were shipped in collection and transport media (Table 3) to the Zavros laboratory (University of Arizona) for processing within 24 hours of collection. Resected PitNET tissues were washed with Dulbeccos Phosphate Buffered Saline (DPBS) supplemented with 1× Penicillin/Streptomycin, 1× Kanamycin and 1× Amphotericin/Gentamycin, minced into smaller pieces, and incubated with digestion buffer (Dulbecco's Modified Eagle Medium/Nutrient Mixture F-12 (DMEM/F12) supplemented with 0.4% Collagenase 2, 0.1% Hyaluronic Acid, 0.03% Trypsin). The tissue was incubated with Accutase (Thermo Fisher Scientific) for a further 5 minutes, and the cells were pelleted and washed with DPBS supplemented with antibiotics. Dissociated cells were then seeded as Matrigel™ domes in culture dishes and overlaid with growth media (Table 4). Media was replaced every 3 days and organoids were harvested for downstream experiments or passaged after 15 days of culture (FIG. 1B).

TABLE 3
Composition of PitNET Tissue Collection and Transport Media

| Component | Concentration | | Supplier |
|---|---|---|---|
| Serum-free defined medium (SFDM) | 100% | | Thermo Fisher Scientific # |
| | | | 10744019 |
| Y27632 (ROCK inhibitor) | 10 | μM | Sigma-Aldrich # Y0503 |
| 1-Glutamine | 2 | mM | Thermo Fisher Scientific # |
| | | | 350-50-061 |
| A83-01 (activin receptor-like kinase (Alk) | 0.5 | mM | R&D Systems # 2939/10 |
| 4/5/7 inhibitor) | | | |
| Penicillin/Streptamycin | 1% | | Thermo Fisher Scientific # |
| | | | 15140122 |
| Kanamycin Sulfate | 1% | | Thermo Fisher Scientific # |
| | | | 15160-054 |
| Gentamycin/Amphotericin | 0.2% | | Fisher Scientific # R01510 |
| CHIR-98014 | 4 | mM | SIGMA # SML1046 |
| Thiazovivin | 2.5 | mM | SIGMA # SML1045 |

TABLE 4
Composition of hPITO Growth Media

| Component | Concentration | | Supplier |
|---|---|---|---|
| Serum-free defined medium (SFDM) | 100% | | Thermo Fisher Scientific # |
| | | | 10744019 |
| B27 (without vitamin A) | 2% | | Thermo Fisher Scientific # |
| | | | 12587010 |
| N2 | 1% | | Thermo Fisher Scientific # |
| | | | 17502048 |
| 1-Glutamine | 2 | mM | Thermo Fisher Scientific # |
| | | | 350-50-061 |
| Penicillin/Streptamycin | 1x | | Thermo Fisher Scientific # |
| | | | 15140122 |
| N-acetyl-l-cysteine | 1.25 | mM | Sigma-Aldrich # A7250 |
| Nicotinamide | 10 | mM | Sigma-Aldrich # N0636 |
| huEGF | 50 | ng/mL | R&D Systems # 236-EG200 |
| A83-01 (activin receptor-like kinase (Alk) | 0.5 | μM | R&D Systems # 2939/10 |
| 4/5/7 inhibitor) | | | |
| SB431542 (p38 mitogen-activated protein | 10 | μM | R&D Systems # 1614/10 |
| kinase inhibitor) | | | |
| IGF1 | 100 | ng/mL | R&D Systems # 291-G1 |
| WNT conditioned media | 50% | | |
| Rspondin conditioned media | 10% | | |
| FGF2 (=basic FGF) | 20 | ng/mL | R&D Systems # 233-FB |
| FGF8 | 200 | ng/mL | R&D Systems # 423-F8 |
| FGF10 | 100 | ng/mL | PeproTech # 100-26 |
| FGF18 | 10 | ng/mL | R&D Systems # 8988-F18 |
| huSHH | 30 | ng/mL | R&D Systems # 1845-SH |
| Noggin | 100 | ng/mL | PeproTech # 250-38 |
| Y27632 | 10 | mM | Sigma-Aldrich # Y0503 |

### 1.4 Immunofluorescence

iPSCs were stained for pituitary specific hormones including luteinizing hormone (LH), follicle-stimulating hormone (FSH), growth hormone (GH), prolactin (PRL), ACTH, cytokeratin 8/18 (CAM5.2), pituitary transcription factor PIT-1 and neuroendocrine marker Synaptophysin (Table 5). Cultures were immunostained for proliferation marker EdU (Click-IT EdU Alexa Fluor 555 Imaging Kit, Thermo Fisher Scientific C10338). Antibody dilutions were determined based on manufacturer guidelines and antibody titration (Table 5). Staining was conducted according to previously published protocols [29, 30]. Briefly, organoids and iPSCs grown in 8-well glass bottom chamber slides were fixed using 3.7% formaldehyde, washed with DPBS, permeabilized with 0.5% Triton X-100 (SIGMA X-100, PBS-T, 20 min at room temperature), washed with 0.01% PBS-T, and blocked with 2% donkey serum (Jackson Immuno Research 017-000-121) for 1 hour at room temperature. After blocking, primary antibodies were added and incubated overnight at 4° C. Slides were washed with 0.01% PBS-T and incubated with secondary antibodies (Table 5) with Hoechst (Thermo Fisher Scientific H1399) for 1 hour at room temperature. Organoids were washed and stored in PBS at 4° C. until microscopic analysis. High content confocal microscopy was performed using the Nikon Ti2-E Inverted Microscope (with a Crest X-Light V2 L-FOV Spinning Disk Confocal) at the University of Arizona Cancer Center, or a ZEISS LSM880 34 channel Laser Scanning Confocal Microscope at University of Arizona Marley Imaging Core. Fluorescence intensity was quantified as the percentage of marker-positive cells versus total cells, using Nikon Elements Software (Version 5.21.05). Proliferation for each iPSC line was quantified as the percentage of EdU-positive cells versus total cells.

TABLE 5
Concentrations of Antibodies Used

| | | |
|---|---|---|
| Antibody | Dilution | Catalog Number |

Immunofluorescence

| | | |
|---|---|---|
| anti-Rabbit PIT1 | 1:50 | Thermo Fisher Scientific # PA5-98650 |
| anti-Rabbit ACTH | 1:250 | Thermo Fisher Scientific # 701293 |
| anti-Rabbit Synaptophysin | 1:100 | Thermo Fisher Scientific # PA5-27286 |
| anti-Mouse CAM5.2 | 1:250 | SIGMA 452M-95 |
| anti-Mouse GH | 1:100 | Novus Biologicals # NB500- |
| | | 364AF647 |
| anti-Mouse FSH | 1:100 | Thermo Fisher Scientific # MIF2709 |
| anti-Rabbit LH | 1:100 | Thermo Fisher Scientific # PA5- |
| | | 102674 |
| anti-Mouse PRL | 1:100 | Thermo Fisher Scientific # CF500720 |
| Alexa Fluor 488 Donkey Anti Rabbit IgG | 1:100 | Thermo Fisher Scientific # A-21206 |
| (H + L) | | |
| Alexa Fluor 488 Goat Anti Rabbit IgG | 1:100 | Thermo Fisher Scientific # A-11034 |
| (H + L) | | |
| Alexa Fluor 647 Donkey Anti Mouse IgG | 1:100 | Thermo Fisher Scientific # A-31571 |
| (H + L) | | |
| Alexa Fluor 633 Goat Anti Mouse IgG | 1:100 | Thermo Fisher Scientific # A-21052 |
| (H + L) | | |

Immunohistochemistry

| | | |
|---|---|---|
| anti-Mouse CAM5.2 | 1:100 | Millipore SIGMA 452M-95 |
| anti-Rabbit Synaptophysin | 1:100 | Thermo Fisher Scientific PA5-27286 |
| anti-Mouse T-PIT | 1:100 | Thermo Fisher Scientific MA5-31431 |
| anti-Mouse ACTH | 1:100 | Thermo Fisher Scientific MA5-13455 |

### 1.5 Nuclear Morphometric Analysis (NMA)

Nuclear Morphometric Analysis (NMA) using treated organoids was performed based on a published protocol that measures cell viability based on the changes in nuclear morphology of the cells using nuclear stain Hoechst or DAPI [31]. Images of organoid nuclei were analyzed using the ImageJ Nuclear Irregularity Index (NII) plugin for key parameters that included cell area, radius ratio, area box, aspect, and roundness. Using the published spreadsheet template [31], the NII of each cell was calculated with the following formula: NII=Aspect−Area Box+Radius Ratio+Roundness. The area vs NII of vehicle-treated cells were plotted as a scatter plot using the template and considered as the normal cell nuclei. The same plots were generated for each condition, and the NII and area of treated cells were compared to the normal nuclei, and classified as one of the following NMA populations: Normal (N; similar area and NII), Mitotic (S; similar area, slightly higher NII), Irregular (I; similar area, high NII), Small Regular (SR; apoptotic, low area and NII), Senescent (LR; high area, low NII), Small Irregular (SI; low area, high NII), or Large Irregular (LI; high area, high NII). Cells classified as SR exhibited early stages of apoptosis, and cells classified as either I, SI or LI exhibited significant nuclear damage. The percentage of cells in each NII classification category were calculated and plotted as a histogram using GraphPad Prism.

### 1.6 Immunohistochemistry (IHC)

PitNET organoids were fixed in 4% paraformaldehyde and embedded in Epredia™ HistoGel™ Specimen Processing Gel according to the Manufacturers protocol (Fisher Scientific HG-4000-012). HistoGel embedded organoids were then paraffin embedded and sectioned on slides at a 5-micron thickness. After deparaffinization and antigen retrieval (Antigen Unmasking Solution, Vector Laboratories H-3300-250), endogenous peroxidase activity was blocked using 0.3% hydrogen peroxide/methanol for 20 min. Slides were blocked with 20% normal goat or horse serum for 20 min at room temperature and incubated with primary antibodies overnight at 4° C. (Table 5). Slides were incubated with biotinylated anti-mouse or anti-rabbit secondary antibodies for 30 minutes. Color was developed with 3,3′-diaminobenzidine (DAB) using the DAB substrate VECTASTAIN Elite Mouse (Vector Laboratories PK-6102) or Rabbit (Vector Laboratories PK-6101). Immunohistochemical slides were dehydrated and mounted using Permount (Fisher Scientific), and images were viewed and captured under light microscopy (Olympus BX60 with Diagnostic Instruments “Spot” Camera; Tokyo, Japan).

### 1.7 Organoid Drug Treatments and Dose Responses

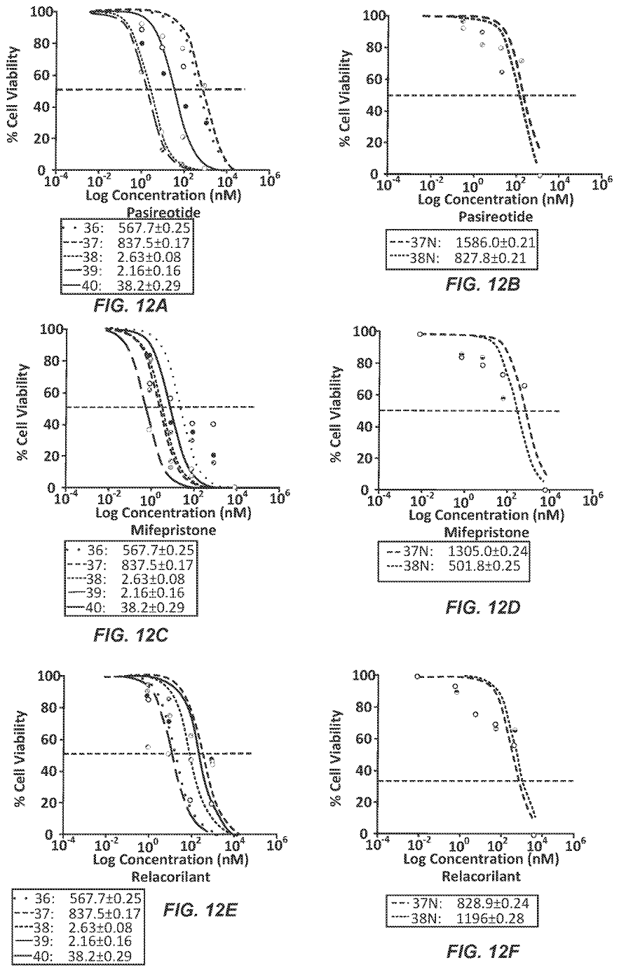

Sonic Hedgehog (SHH) was withdrawn from the organoid growth media 16 hours prior to treatment. The experimental treatment groups included: vehicle (Veh), mifepristone (500 nM, Selleckchem S2606), GANT61 (5 μM, Stemcell Technologies 73692), ketoconazole (Keto, 10 μM, Selleckchem S1353), pasireotide (Pas, 100 nM, Target Mol TP2207-SB001), octreotide (Oct, 100 nM, Selleckchem P1017), mifepristone plus GANT61, mifepristone plus Keto, mifepristone plus Pas, relacorilant (500 nM, Corcept Therapeutics CORT125134), relacorilant plus Pas, relacorilant plus Oct, or dexamethasone (Dexa, 100 nM, SIGMA S4902). In the combination treatments, cultures were pretreated with the GANT61, Keto, Pas or Oct for 2 hours prior to treatment with mifepristone or Rela. PitNET tissue derived organoids hPITO36, hPITO37, hPITO38, hPITO39, and hPITO40 were treated with the following experimental conditions: Vehicle (1), pasireotide (2), relacorilant (3), relacorilant & pasireotide (4), mifepristone (5), mifepristone & pasireotide (6). The IC₅₀ values used for each hPITO line for each drug were determined based on dose responses (FIG. 12). All organoid cultures were treated for 48 hours prior to analysis.

### 1.8 ACTH Enzyme-Linked Immunosorbent Assay (ELISA)

Organoid conditioned media was collected and ACTH secretion using an ELISA according to the manufacturer's protocol (Novus Biologicals Human ACTH ELISA Kit NBP2-66401). The optical density was measured at a wavelength of 450 nm, and ACTH concentration (pg/mL) was interpolated using a standard curve with a 4-parameter logistic regression analysis using GraphPad Prism (v9.3.1).

1.9 Quantitative RT-PCR (qRT-PCR)

RNA was extracted from PitNET tissue or iPSC generated organoids using TRI Reagent® (Molecular Research Center Inc TR118) and the High Capacity cDNA Reverse Transcription Kit (Applied Biosystems, Foster City, CA) was used for cDNA synthesis of RNA following the recommended protocol. For each sample, 60 ng RNA was reverse transcribed to yield approximately 2 μg total cDNA that was then used for the qRT-PCR. Pre-designed real-time polymerase chain reaction assays were purchased for the following genes: TPit (Thermo Fisher Scientific 4331182 Hs00193027), SF1 (Thermo Fisher Scientific 4331182 Hs00610436), SSTR2 (Thermo Fisher Scientific 4331182 Hs00265624_s1), SSTR5 (Thermo Fisher Scientific 4331182 Hs00990407_s1), POMC (Thermo Fisher Scientific 4331182 Hs01596743_ml), hPRT (Thermo Fisher Scientific 4331182 Hs02800695_ml), and FKBP5 (Thermo Fisher Scientific 4331182 Hs01561006_m1). Polymerase chain reaction amplifications were performed in a total volume of 20 μL containing 20× TaqMan Expression Assay primers, 2× TaqMan Universal Master Mix (Applied Biosystems, TaqMan® Gene Expression Systems), and cDNA template. Each polymerase chain reaction amplification was performed in triplicate wells in a StepOne Real-Time PCR System (Applied Biosystems) by using the following conditions: 500C 2 minutes, 95° C. 10 minutes, 95° C. 15 seconds (denature) and 60° C. 1 minute (anneal/extend) for 40 cycles. Fold change was calculated as the following: (Ct-Ct high)=n target, 2ntarget/2nHPRT=fold change where Ct=threshold cycle. The results were expressed as average fold change in or differential gene expression relative to control, with HPRT used as an internal control according to Livak and Schmittgen [32].

### 1.10 Spectral Flow Cytometry Using the Cytek® Aurora

The multicolor flow cytometry antibody panel was designed using the Cytek® Full Spectrum Viewer online tool to calculate the similarity index. Organoids were harvested in cold Serum-Free Defined Medium media and incubated with Zombie (BioLegend 423107, 1:100) for 15 minutes in the dark at room temperature, followed by incubation with fluorochrome-conjugated/unconjugated primary surface or cytoplasmic antibodies (Table 6) at 4° C. for 30 minutes. Cells were then washed with Cell Staining Buffer (BioLegend 420-201) and incubated at 4° C. for 30 minutes with secondary antibodies (Table 6). Cells were fixed using Cytofix/Cytoperm™ Fixation/Permeabilization Solution (BD Biosciences 554714) and washed with Fixation/Permeabilization wash buffer according to the recommended protocol. Intracellular markers were then labeled with fluorochrome-conjugated/unconjugated primary antibodies at 4° C. for 30 minutes, washed and incubated with secondary antibodies at 4° C. for 30 minutes (Table 6). UltraComp eBeads™, Compensation Beads (Thermo Fisher Scientific 01-2222-42) were stained with the individual antibodies and used as single stain control for compensation and gating. For Zombie dye single cell control, ArC™ Reactive Beads (Thermo Fisher Scientific A10346A) were stained with Zombie and ArC™ negative beads (Thermo Fisher Scientific A10346B). Data was acquired using the Cytek® Aurora, analyzed using Cytobank Premium software (Beckman Coulter).

TABLE 6
Antibodies Used for Flow Cytometric Analysis

| Marker | Fluorophore | Antibodies | Secondary Antibodies |
|---|---|---|---|
| ACTH | AF532 | Novus Biological # NBP2- | — |
| | | 34529AF532 | |
| LH Beta | DyLight 405 | Novus Biological # NBP2- | — |
| | | 47733V | |
| PRL | PE Cy7 | Novus Biological # NBP2- | — |
| | | 73609PECY7 | |
| FSH | AF350 | Novus Biological # NBP2- | — |
| | | 47735AF350 | |
| GH | AF750 | Novus Biological # NBP2- | — |
| | | 54352AF750 | |
| TPIT1 | DyLight 650 | Novus Biological # NBP2- | — |
| | | 74469C | |
| SF1 | AF594 | Novus Biological # | — |
| | | 74338AF594 | |
| TSH | BV 605 | Novus Biological # NBP2- | Biolegend # 405327 (anti- |
| | | 52574 | Mouse) |
| CK20 | AF700 | Novus Biological # NBP2- | — |
| | | 42616AF700 | |
| PanCK | DyLight 755 | Novus Biological # NBP2- | — |
| | | 33200IR | |
| Alpha | BV480 | Novus Biological # NBP3- | Jackson Immuno Research # |
| hcg | | 11024 | 109-685-170 (anti-Goat) |
| Ki67 | BV711 | Biolegend # 350515 | — |
| SOX2 | PacificBlue | Biolegend # 656111 | — |
| Nestin | APC | Thermo Fisher # MA5-23650 | — |
| CD133 | PEDazel 594 | Biolegend # 141211 | — |
| Vimentin | Percp/Cy5.5 | Novus Biological # MAB 2105 | Biolegend # 405424 (anti-Rat) |
| CXCR4 | PE/Cy5 | Biolegend # 3066507 | — |
| CD73 | BUV737 | BD Biosciences # 612812 | — |
| CD90 | BUV496 | BD Biosciences # 741160 | — |
| CD105 | BUV661 | BD Biosciences # 750036 | — |
| KLF4 | DyLight 550 | Novus Biological # NBP1- | — |
| | | 21047R | |
| PIT1 | PE/AF 610 | Thermo Fisher # PA5 98650 | Thermo Fisher # A20981 (anti- |
| | | | Rabbit) |
| Nanog | AF555 | BD Biosciences # 560260 | — |
| CD31 | BV510 | BD Biosciences # 563454 | — |
| CD45 | BV786 | BD Biosciences # 634555 | — |
| SSTR5 | FITC | Novus Biological # FAB4448F | — |
| SSTR2 | Percp | Novus Biological # FAB4224C | — |
| Zombie | UV | Biolegend # 423107 | — |

### 1.11 Statistical Analysis

Data collected from each individual study used 4 in vitro experimental or biological replicates unless otherwise stated. Results were analyzed as the mean±the standard error of the mean (SEM), and data represented as violin or scatter plots, and significance tested using Microsoft Excel and GraphPad Prism (v9.3.1).

### 2. Results

2.1 IPS C-Derived Pituitary Organoids Expressing Somatic Mutations Reveal Corticotroph PitNET Pathology Consistent with CD

Extensive research has revealed the role of somatic mutations in the development of CD adenomas [27, 33]. To study these mechanisms, we generated human pituitary organoids developed from iPSCs. With reference to a published protocol using embryonic stem cells [34] together with the knowledge of the growth factor and genetic regulation of pituitary gland development [35], we optimized our approach for the development of pituitary organoids from human blood-derived iPSCs for gene editing (FIG. 1A). The iPSC lines were generated to express known somatic mutations in iPSC^{USP48} (Gene ID: 84196; M415I (G1245A) and M415V (A1243G)), or iPSC^{USP8} (FIG. 1A, FIG. 11A) [25, 33, 36-41]. USP48 mutations included either M415V or M415I, designating iPSC^{USP48MV} and iPSC^{USP48MI} respectively. Successful gene editing was validated using primer specific visualization of a change in the restriction pattern at the site of interest (FIGS. 11B, C). Brightfield images showed morphological variations between the control (iPSC^{ctrl}) and mutant iPSCs as early as day 4 and at day 15 (FIG. 1A). Morphological changes and proliferation were obvious when iPSCs were embedded in Matrigel™ to generate organoids (FIG. 1A).

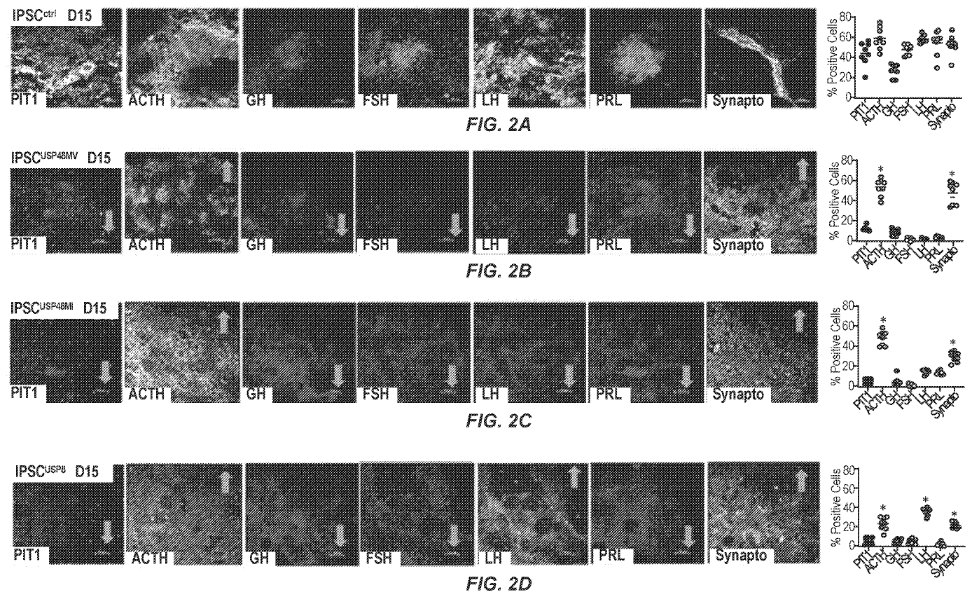

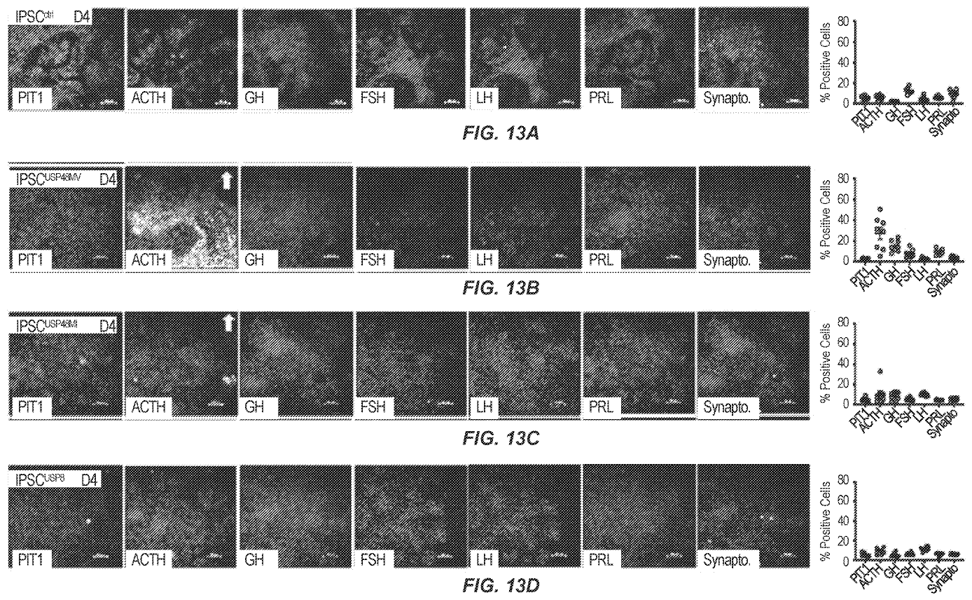

Expression of PIT1 (pituitary-specific positive transcription factor 1), ACTH (adrenocorticotropic hormone), GH (growth hormone), FSH (follicle-stimulating hormone), LH (luteinizing hormone), PRL (prolactin) and synaptophysin (synapto) with co-stain Hoechst (nuclei, blue) was measured by immunofluorescence using chamber slides collected at days 4 (D4, FIGS. 13A-D) and 15 (D15, FIGS. 2A-D) of the differentiation schedules. While pituitary tissue that was differentiated from control iPSCs (iPSC^{ctrl}) expressed all major hormone-producing cell lineages (FIG. 2A), there was a significant increase in the expression of ACTH and synaptophysin with a concomitant loss of PIT1, GH, FSH, LH and PRL in iPCSs expressing mutated USP48 and USP8 (FIGS. 2B, C, D). Immunofluorescence of iPSCs collected at day 4 of the differentiation schedule revealed no expression of PIT1, ACTH, GH, FSH, LH and PRL in iPSC^{ctrl} (FIG. 13A). Unexpectedly, ACTH expression was increased in iPSC^{USP48} at day 4 (FIGS. 13B, C).

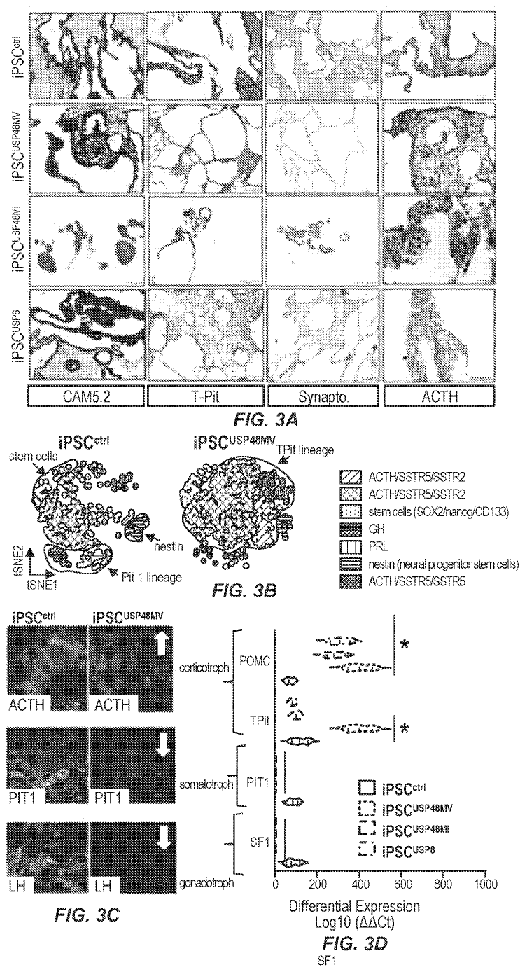

Immunohistochemistry of formalin fixed and paraffin embedded iPSC^{ctrl}, iPSC^{USP48}, and iPSC^{USP8} expressed CAM5.2, T-PIT, synaptophysin and ACTH (FIG. 3A). The expression pattern of corticotroph PitNET specific markers was analyzed using Cytek™ Aurora spectral flow cytometry (FIG. 3B). The location of cells that were found in each cluster based on the highly expressed antigens are shown in the representative heatmap t-SNE maps (FIG. 3B). Compared to iPSC^{ctrl} organoids, iPSC^{USP48} and iPSC^{USP8} contained higher numbers of cells expressing T-Pit cell lineage and ACTH/SSTR2/SSTR5 expressing cells (representative t-SNE map of iPSC^{USP48MV} shown in FIG. 3B). In addition, iPSC^{ctrl} organoids expressed a higher percentage of Pit1 cell lineage, GH and PRL positive cells (FIG. 3B).

The differential expression of the major transcription factors was correlated with the classification of PitNETs based on immunofluorescence (FIG. 3C, D). The iPSCcrtl line expressed transcription factors TPit (TBX19), PIT1 (POUIF1) and SF1 that corresponded to the differentiation of corticotrophs, somatotrophs and gonadotrophs as documented by positive immunofluorescence staining (FIGS. 3C, D). The iPSC lines expressing mutations in USP48MV exhibited significantly elevated transcription factor T-Pit consistent with the skewed differentiation in the corticotroph cell lineage (FIGS. 3C, D). Transcription factors PIT1 (somatotrophs) and SF1 (gonadotroph) were significantly decreased in the cultures of iPSC lines expressing USP48MV compared to iPSC^{ctrl} (FIG. 3C, D).

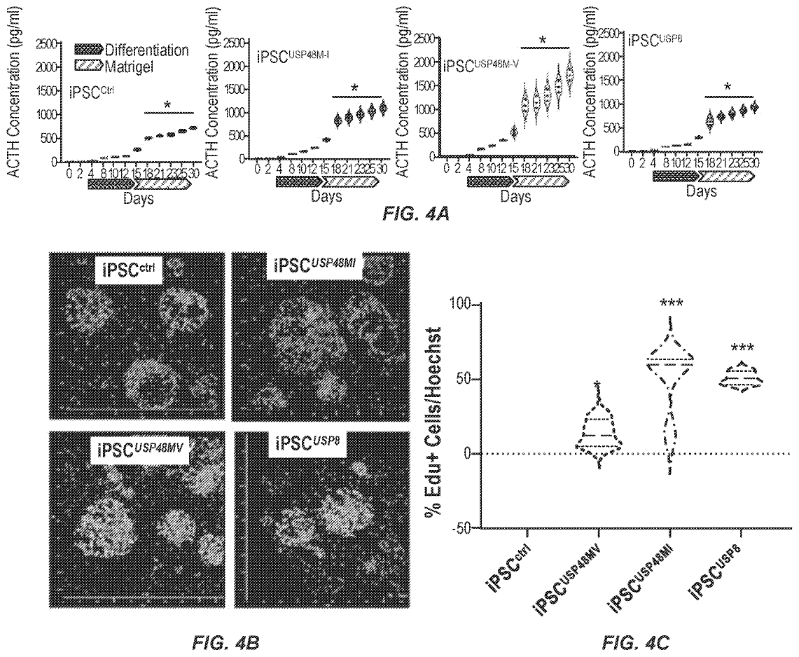

Culture conditioned media was collected during the differentiation schedule of the iPSCs and analyzed for ACTH secretion by ELISA (FIG. 4A). Compared to control lines, iPSC lines expressing mutated USP8 and USP48 secreted significantly greater concentrations of ACTH earlier in the differentiation schedule (FIG. 4A). Differentiated iPSCs were then collected at D15 of the culture schedule and embedded into Matrigel™. Proliferation was quantified by EdU uptake of the cells within the pituitary organoids (FIG. 4B). Compared to control pituitary organoids iPSC^{ctrl}, organoids expressing mutations in USP8 (iPSC^{USP8}), and USP48 (iPSC^{USP48MV} and iPSC^{USP48MI}) expressed significantly elevated Edu+proliferating cells (FIG. 4C). Collectively, these data show that iPSCs genetically engineered to express somatic mutations relevant to the development of CD exhibit corticotroph PitNET pathology and function in vitro.

2.2 Genetically Engineered iPSCs Expressing Somatic Mutations Relevant to CD Identify Differential Effects of Relacorilant and Mifepristone on SSTR Expression, and Tumor Cell Proliferation and Apoptosis

The treatment of iPSC organoids with mifepristone or relacorilant resulted in a significant induction in the expression of SSTR2 and 5 (FIG. 5A, B). Mifepristone led to a significantly greater induction in SSTR2 expression when compared to relacorilant (FIG. 5A). In contrast, relacorilant induced greater SSTR5 expression (FIG. 5B).

To identify the role Hedgehog signaling as a mediator of mifepristone or relacorilant regulated SSTR expression, iPSC^{ctrl} organoids were used in combination with inhibitors GANT61 (GLI inhibitor) and ketoconazole (a SMO inhibitor that also inhibits cortisol synthesis) (FIG. 5C, D). Mifepristone significantly induced the differential expression of both SSTRs 2 and 5, and this increase was reduced with GANT61 pretreatment of the organoid cultures (FIG. 5C). In contrast to the inhibitory effect of GANT61 on mifepristone-induced SSTR expression, ketoconazole pretreatment had no effect on this induction (FIG. 5C), GANT61 and ketoconazole alone had no effect on the differential expression of SSTRs 2 and 5, while dexamethasone, a known GR agonist, significantly decreased expression of these receptors (FIG. 5C). Similar responses were observed with relacorilant with or without GANT61 and ketoconazole (FIG. 5D). These results suggest that mifepristone and relacorilant induce SSTR2 and SSTR5 via a SMO-independent non-canonical Hh signaling pathway.

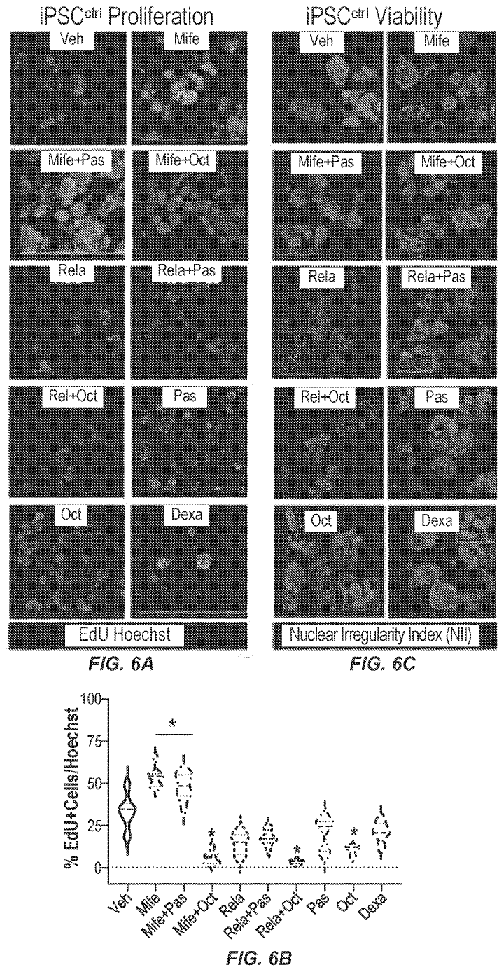

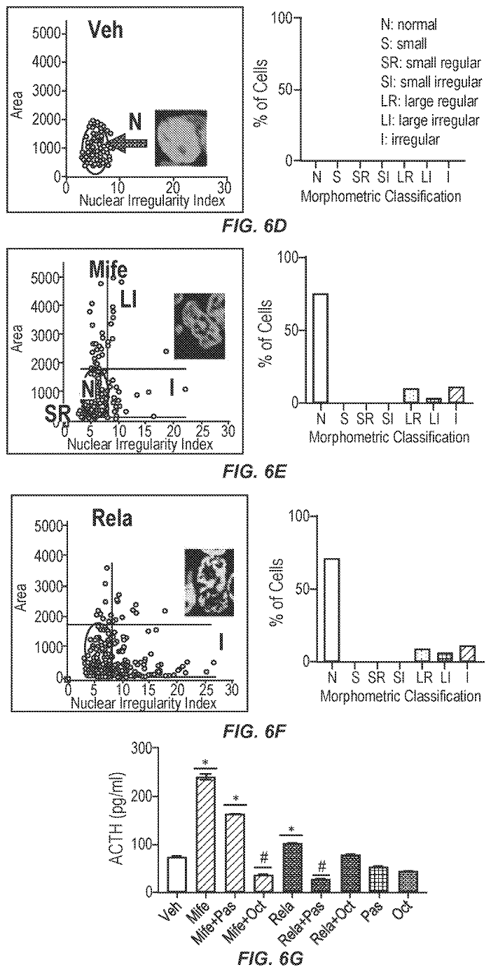

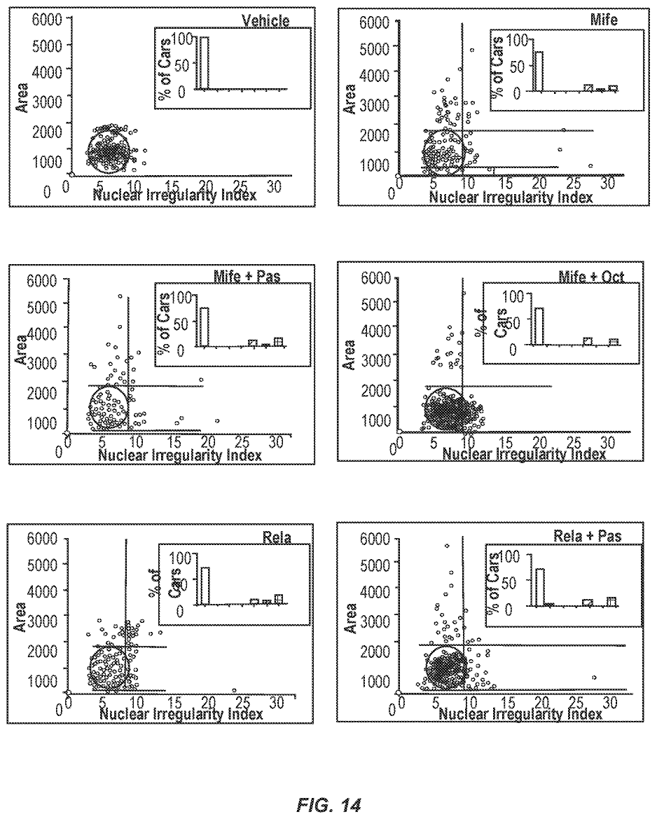

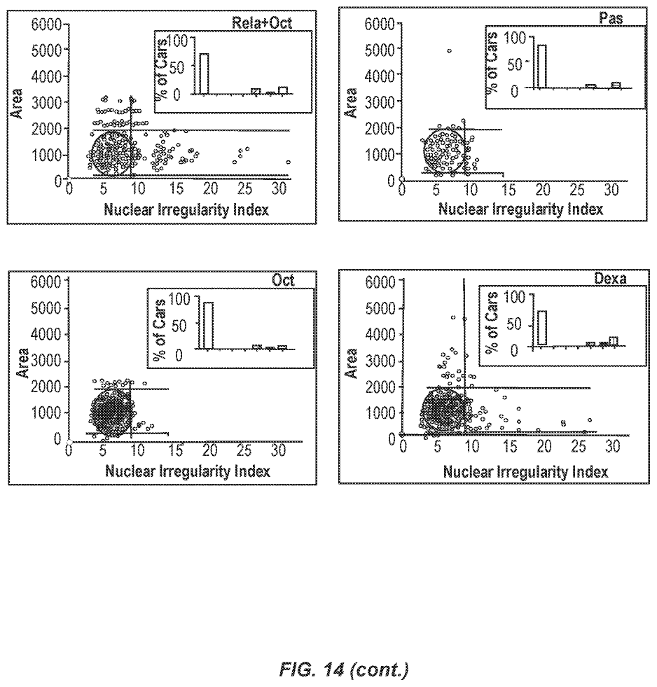

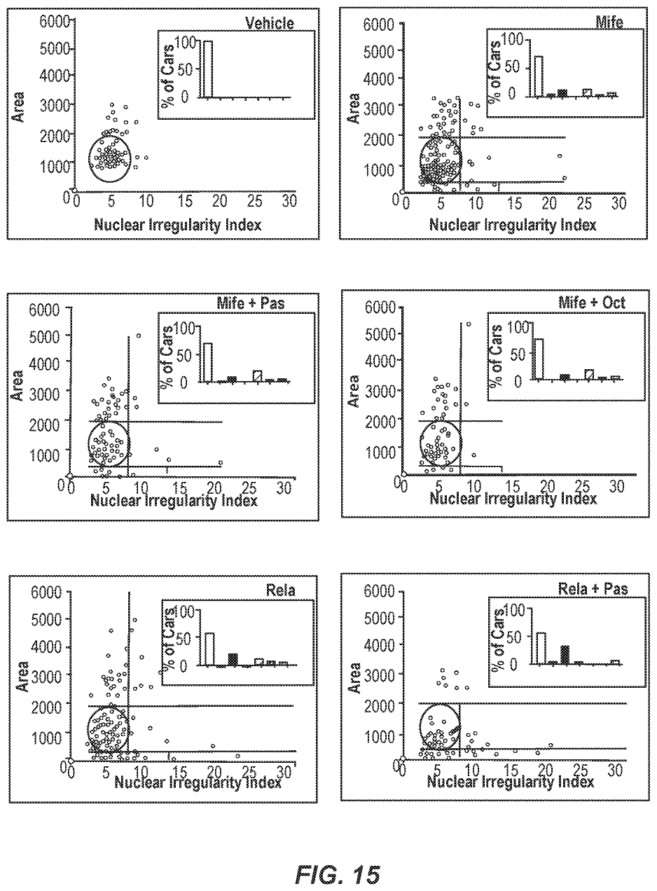

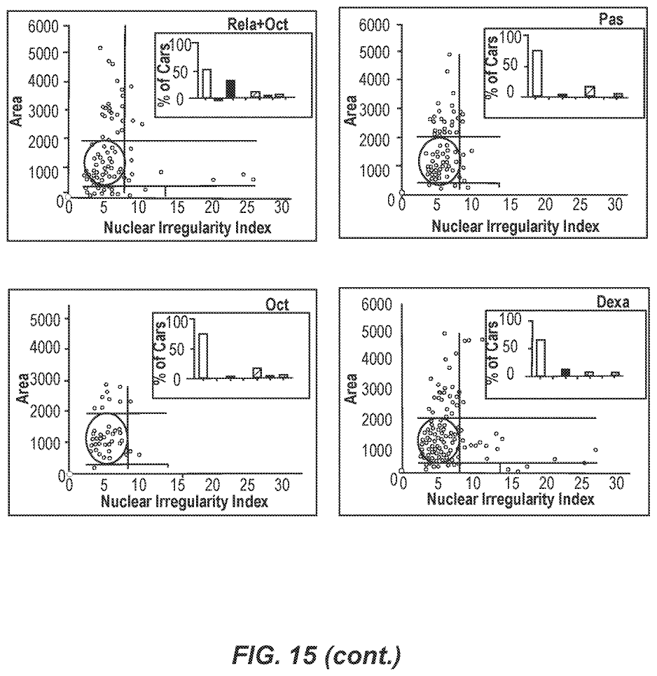

Analysis of proliferation using EdU uptake in iPSC^{ctrl} and iPSC^{USP48MV} organoid cultures showed that mifepristone induced a significant increase in cell proliferation (FIG. 6A, B and FIG. 7A, B). While the proliferative response to mifepriston was inhibited by pretreatment with Oct, Pas had no effect (FIG. 6A, B and FIG. 7A, B). While a similar response was observed in relacorilant treated cultures with pretreatment with Pas or Oct, relacorilant alone did not induce organoid proliferation (FIG. 6A, B and FIG. 7A, B). The nuclear irregularity index (NII) was measured based on the quantification of the morphometric changes in the nuclei in response to the treatment groups in the iPSC^{ctrl} (FIG. 6C-F, FIG. 14) and iPSC^{USP48MV} organoids (FIG. 7C-F, FIG. 15). While relacorilant induced a significant expression pattern of nuclear morphology consistent with increased apoptotic cells in the iPSC^{USP48MV} cultures (FIG. 7C-F, FIG. 15), this was not observed in the iPSC^{ctrl} organoids (FIG. 6C-F, FIG. 14). The magnitude of the apoptotic response to relacorilant was not observed with mifepristone in either the iPSC^{ctrl} (FIG. 6C-F, FIG. 14) or iPSC^{USP48MV} (FIG. 7C-F, FIG. 15) organoids.

Within the iPSC^{ctrl} and iPSC^{USP48MI} organoid cultures, the magnitude of ACTH secretion induced by mifepristone was significantly greater than the effect of relacorilant (FIGS. 6G and 7G). Pas and Oct significantly reduced ACTH secretion in response to mifepristone, although the inhibition by octreotide was greater that pasireotide (FIG. 6G and FIG. 7G). Pasireotide reduced hormone secretion in combination with relacorilant at a greater magnitude compared to that of octreotide plus relacorilant in iPSC^{ctrl} and iPSC^{USP48MV} organoid cultures (FIG. 6G and FIG. 7G).

Heatmap dot plots obtained after tSNE analysis of iPSC^{USP48MV} organoids showed the relative expression of the SSTR2, SSTR5, ACTH and zombie positive cells in the different phenotypic clusters in response to relacorilant and mifepristone (FIG. 8). Quantification based on the gated identified cell clusters revealed that both relacorilant and mifepristone induced a significant increase in percentage of SSTR2 (FIGS. 8A, E) and SSTR5 (FIGS. 8B, F) positive cells. However, the magnitude of mifepristone induction of SSTR2 was significantly greater than that of relacorilant (FIGS. 8A, E). Similarly, the magnitude of relacorilant induction of SSTR5 was significantly greater than that of mifepristone (FIGS. 8B, F). In contrast to relacorilant, mifepristone led to a significant induction in the number of ACTH positive cells (FIG. 8C, G). Relacorilant did not significantly increase ACTH, and Pas+relacorilant significantly reduced ACTH expression in cultures (FIG. 8C, G). Relacorilant, or relacorilant plus Pas, clearly induced iPSC^{USP48MV} cell death as measured by the significant increase in Zombie positive cells that co-expressed ACTH and stem cell markers, a response not observed with mifepristone (FIG. 8D, H).

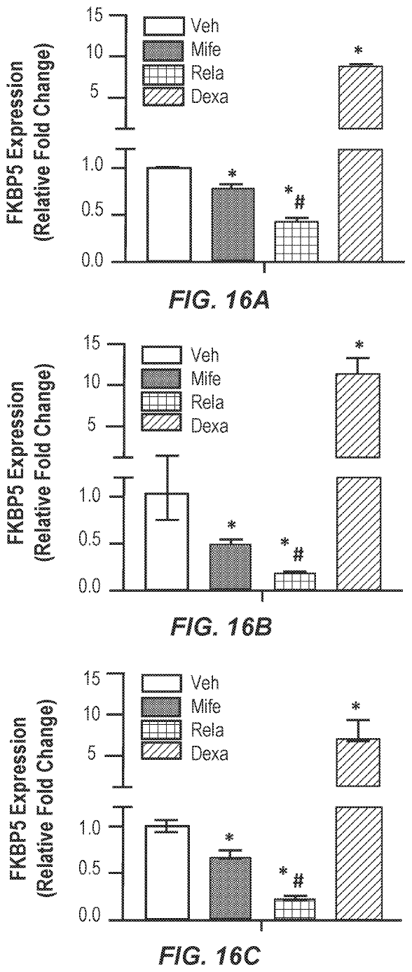

To assess GR modulation, the GR target gene FKBP5 was measured in the PitNET organoids. While mifepristone and relacorilant significantly reduced the differential expression of FKBP5, dexamethasone caused a significant induction in gene expression (FIG. 16A-C). Compared to mifepristone, the magnitude of FKBP5 inhibition was significantly greater in response to relacorilant (FIG. 16A-C). This confirmed GR modulation in the organoid cultures in response to mifepristone and relacorilant.

Collectively, these data demonstrate that, compared to relacorilant, mifepristone preferentially induced the expression of SSTR2, ACTH secretion and PitNET organoid proliferation. In contrast, relacorilant predominantly induced SSTR5 expression and PitNET organoid apoptosis.

3.3 Relacorilant Induces Tumor Cell Death and Reduces ACTH in Combination with Pasireotide in Organoids Generated from CD Patient PitNET's

As part of a previous study, we generated an initial biobank of organoids generated from CD patient PitNETs. Human PitNET tissue was harvested during endoscopic transsphenoidal pituitary surgery from 40 patients to generate organoids. These cultures are referred to as human PitNET derived organoids (hPITOs). Table 1 summarizes the neuropathology reports and clinical diagnosis from cases used to generate hPITOs 37, 38, 39, 40 reported in the current study. Bright-field microscopy images of hPITOs that were generated from corticotroph PitNETs from patients diagnosed with CD revealed morphological diversity, including size, density, and regularity, among the organoid lines between individual patients and between Crooke's cell adenoma and sparsely granulated subtypes (FIG. 9A), Immunofluorescence confocal microscopy showed the expression of ACTH and CAM5.2 (cytokeratin 8/18) pituitary corticotroph markers (representative image of Crooke's cell hPITO37 shown in FIG. 9B; a video recording of such expression was also obtained (not shown).

To further test the similarity in cell lineages identified between the organoid line and the patient's tumor, we compared the immunohistochemistry from the neuropathology report to the expression pattern of pituitary tumor specific markers that were measured using Cytek™ Aurora spectral flow cytometry (FIG. 9C). Flow cytometric analysis using Cytobank revealed that hPITOs derived from patients with CD expressed increased stem and progenitor cell markers including CXCR4, SOX2, and CD133 (FIG. 9C). Using a gating strategy for the TPit positive cells, we identified ACTH positive cell populations co-expressing SSTR2 and SSTR5 (FIG. 9C). Diversity in the differential expression of SSTR2 and SSTR5 among the different hPITO lines was observed when SSTR2 and SSTR5 gene expression was measured (FIG. 9D).

Organoid line hPITO37 (Crooke's cell adenoma subtype) expressed significantly lower SSTR2 and SSTR5 expression levels compared to the other cultures (FIG. 9D) and was insensitive to Pas (FIG. 12). Therefore, we used the hPITO line to identify the differential effects of mifepristone and relacorilant on receptor expression and cell viability. Heatmap dot plots obtained after tSNE analysis of hPITO cultures showed the relative expression of the SSTR2, SSTR5, ACTH and zombie positive cells in the different phenotypic clusters in response to relacorilant and mifepristone (FIG. 9E-L). Quantification based on the gated identified cell clusters revealed that both relacorilant and mifepristone induced a significant increase in percentage of SSTR2 and SSTR5 positive cells (FIG. 9E-H). However, the magnitude of mifepristone induction of SSTR2 was significantly greater than that of relacorilant (FIG. 9E, F). While both relacorilant and mifepristone induced a significant increase in percentage SSTR5 positive cells, the magnitude of relacorilant induction of SSTR5 was significantly greater than that of mifepristone (FIG. 9G, H). In contrast, mifepristone significantly increased the number of ACTH expressing cells in culture, a response that was not inhibited by Pas pretreatment (FIG. 9I, J). Relacorilant clearly induced hPITO cell death as measured by the significant increase in Zombie positive cells in response to relacorilant or relacorilant plus Pas, a response not observed with mifepristone (FIG. 9K, L). In contrast to mifepristone, relacorilant also induced significant cell death in cell populations expressing stem cell markers SOX2, CXCR4 and nestin, and an epithelial/mesenchymal hybrid cell population that co-expressed CK20, vimentin and CXCR4 (FIG. 9K, L). Therefore, relacorilant sensitizes CD PitNET tissue derived organoids to pasireotide and induces cell death in several different morphologically diverse cell populations.

Lack of effective medical therapies targeted directly to the corticotroph PitNETs is potentially attributed to the lack of human patient-relevant model systems that recapitulate the cellular complexity of the tumor. Our studies contribute to overcoming this challenge by generating a PitNET organoid model system that is applied to identifying the mechanisms of action of mifepristone and relacorilant (alone and in combination with other medical therapies for Cushing disease). As part of our studies, PitNET organoids were generated from: 1) CRISPR-Cas9 gene editing of patient iPSCs, and 2) CD patient corticotroph PitNETs (hPITOs). Using these advanced in vitro culture systems, we demonstrated that SSTR2 and SSTR5 are targets of Hedgehog transcription effector Gli1 and this response is attenuated by activation of the GR pathway. In these experiments, we have shown that mifepristone and relacorilant exerted differential effects on the induction of SSTR2 and SSTR5 expression, ACTH secretion and PitNET organoid proliferation and apoptosis.

While both mifepristone and relacorilant significantly induced the expression of SSTR2 and SSTR5, the magnitude of induction of these receptors was different. While mifepristone predominantly induced SSTR2 expression, relacorilant predominantly induced SSTR5. The SSTRs expressed on the surface of corticotroph PitNETs have often been targeted for the treatment of CD [19]. Multiple studies suggest that SSTR5 is consistently overexpressed in corticotroph PitNETs [19], however, treatments with somatostatin analogues (octreotide and pasireotide) are designed without considering variations in receptor subtype expression among individual patients with CD. We clearly show the presence of variation in the SSTR2 and SSTR5 expression amongst individual patients with CD (FIG. 9), and, importantly, SSTR5 was significantly upregulated by relacorilant and subsequently sensitized organoid tumor cells to suppression of ACTH by pasireotide (FIG. 9). Our findings are consistent with the knowledge that pasireotide has high-affinity binding to SSTR5 [20]. In fact, pasireotide monotherapy normalizes cortisol in up to 42% of patients with CD but assumes that all patients express this receptor subtype [21]. Combination treatment using pasireotide administered together with cabergoline and ketoconazole may increase the efficacy of the somatostatin analogue, but there is no clear consideration for changes in SSTRs during treatment [21]. Therefore, our findings suggest that treating patients with CD with relacorilant may increase the efficacy of pasireotide (or, potentially, endogenous somatostatin) suppression of ACTH secretion from corticotroph PitNETs.

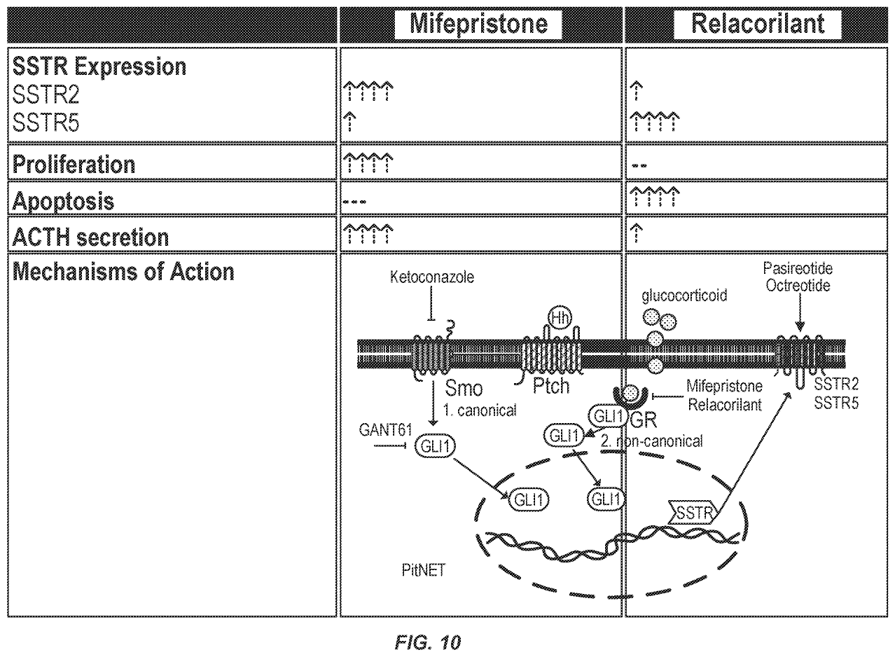

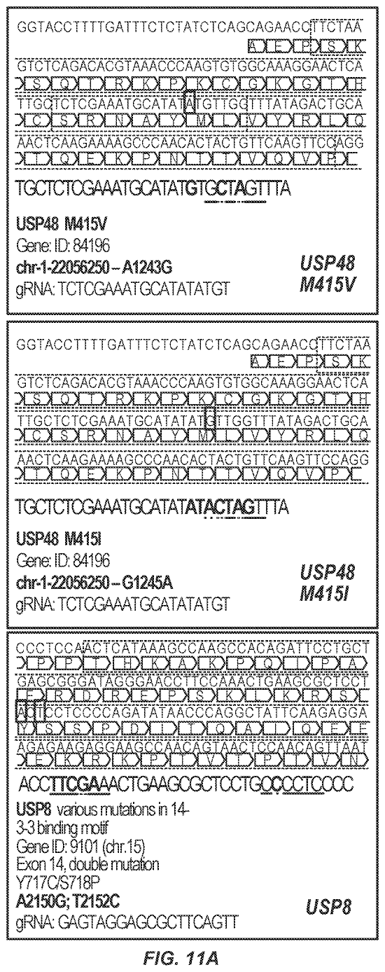

Our studies showed that Hedgehog (Hh) signaling mediates SSTR2 and SSTR5 expression induced by mifepristone and relacorilant through a non-canonical Hh signaling pathway. In the iPSC organoids, GLI inhibitor GANT61 inhibited both mifepristone and relacorilant stimulated SSTR2 and SSTR5. The SMO inhibitor ketoconazole (which also inhibits cortisol synthesis) failed to block the induction of both SSTR2 and SSTR5 by both compounds. Upon ligand binding to the transmembrane receptor Patched-1 (PTCH1), the repression of PTCH1 on transmembrane transducer SMO is released. De-repressed SMO triggers the activation of GLI zinc finger transcription factors GLI1, GLI2 and GLI3. Activated GLI1 protein translocates into the nucleus, where several target genes are regulated [42]. Our studies suggest that within the corticotroph PitNETs, SSTR2 and SSTR5 are transcriptional targets of GLI. The failure of the SMO inhibitor ketoconazole to inhibit mifepristone and relacorilant induction of SSTR expression suggests that the signaling pathway is non-canonical (FIG. 10). Non-canonical Hh signaling pathways have been classified as 1) Type I pathways involving signaling through PTCH1 independent of SMO, 2) Type II pathways involving signaling through SMO independent of GLI, and 3) SMO-independent activation of GLI such as that observed in the current study [42, 43]. In support of our findings, crosstalk between GLI and GR (NR3C1) signaling has been reported in T cell acute lymphoblastic leukemia [44], In addition, studies in the adult stomach demonstrated that the Hh pathway is critical for the regulation of somatostatin and SSTR signaling [45, 46]. While Hh signaling is known to be essential during the embryonic development of the pituitary and in the adult gland [47], this is the first report demonstrating that Hh signaling via Gli1 regulates the expression of SSTR2 and SSTR5 in PitNET organoids.

In contrast to mifepristone, relacorilant induced PitNET cell death in treated organoid cultures without significant stimulation of ACTH secretion. In concurrence with the organoid in vitro studies, tumor regression in two patients with macroadenomas treated with relacorilant for three months have been reported in an initial study based on standard of care imaging of the pituitary gland [17]. This unexpected finding is further being investigated in an ongoing Phase 3 study of relacorilant in patients with Cushing syndrome (GRACE Study, NCT03697109). Such macroadenoma regressions have not been observed with mifepristone. Based on the negative feedback mechanism regulating the hypothalamic-pituitary-adrenal (HPA) axis, we propose the following mechanisms to explain the differential effects between mifepristone and relacorilant. First, mifepristone predominantly induced SSTR2 expression. However, unlike relacorilant, mifepristone treatment significantly increased ACTH secretion from PitNET both in vivo and in vitro (FIG. 10). Our organoid experiments are supported by patient data showing that mifepristone increases ACTH [13, 14], with a concomitant increase in cortisol secretion without tumor growth [19, 48]. In contrast, relacorilant predominantly induced SSTR5 expression without an increase in ACTH secretion and tumor cell death (FIG. 10). A plausible explanation for PitNET regression in response to relacorilant treatment may be the known heterodimerization between SSTR2 and SSTR5 that occurs following the selective activation of SSTR2 but not human SSTR5 or concurrent stimulation [49, 50]. Heterodimerization between SSTR2 and SSTR5 leads to an increased recycling rate and a greater propensity of SSTR2 to induce tumor growth inhibition [50, 51]. Thus, in vivo, the significant effect of mifepristone on SSTR2 and increased receptor signaling offsets the proliferative effects of this antagonist and may explain the lack of tumor growth in treated patients. Relacorilant may also induce PitNET apoptosis independently of SSTR activation (FIG. 10). In support of this notion, studies in several cancers that are driven by the dysregulation of these receptors, including leukemia, breast, prostate, lung and ovarian cancers, show promise of targeting the GR as a strategy for combination treatment [52, 53]. In addition, relacorilant induces apoptosis in vitro using pancreatic and ovarian cancer cell lines [54]. The pro-apoptotic effects of GR modulators are tissue specific and may be a result of targeting the apoptotic/cycle signaling pathways and genes such as Bcl2 [55]. The mechanism of action of relacorilant on PitNET cell apoptosis requires further investigation. Collectively, these studies support broader investigation of relacorilant alone or in combination with somatostatin analogs as pre- or post-operative medical treatment in patients with CD.

While many investigators have proposed using organoids in personalized medicine for the targeted treatment of cancers [29, 56-58], this study is the first to execute this approach for the potential treatment of corticotroph type PitNETs associated with the development of CD. Existing medical therapy for CD remains suboptimal with negative impact on health and quality of life, including the considerable risk of therapy resistance and tumor recurrence. Our data demonstrate that organoids derived from corticotroph type PitNETs consist of differentiated cell lineages, stem/progenitor cells, and stromal cells that replicate much of the patient's own tumor pathology and function clearly documented by extensive high-throughput flow cytometry. Previously published in vitro experiments in the field of CD research were performed using pituitary cell lines or spheroids, aggregates and tumoroids that do not replicate the primary pituitary tumor microenvironment due to cell transformation and/or unphysiological 2D culture conditions [59-61].

Pituitary research has largely been conducted using cell culture techniques using rat (GH3) or mouse (AtT20) pituitary-like cell lines lacking a multicellular identify reflective of the PitNET [62, 63]. Recent advances have led to the development of pituitary tissue generated organoids, but these are limited to the use of transgenic mouse models as the source [64-66]. Nys et al. [67] reported the generation of human pituitary tumor organoids from a single stem cell as claim of true organoids due to the clonality. Unlike our studies, the multicellular complexity was not validated by the protein expression or hormone secretion from pituitary cell lineages in these cultures [67].

It is fundamental to note that according to the National Cancer Institute (NCI, NIH), an ‘organoid’ is defined as “A tiny, 3-dimensional mass of tissue that is made by growing stem cells (cells from which other types of cells develop) in the laboratory.” [68]. While our cultures begin with both single and 3-4 cell clusters isolated from the native CD patient PitNET tissue, these clusters begin a process of ‘budding’, growth and differentiation as documented by videos and by the comprehensive spectral flow cytometry analysis documenting functional cell lineages. Importantly, the results show a different process than what would be an expected observation for the formation of a tumoroid which is the migration and adhesion of initially separate cells to form an aggregate. There are the reports of mouse nonadherent spheres with stem/progenitor characteristics [69], and human embryonic stem cell generated spheroids or patient derived tumoroids that also lack a multicellular identify and consist of poorly differentiated cells [70-75]. Therefore, the findings reported here are significant because we offer an advanced in vitro approach that reveals cell populations expressing stem cell markers that potentially contribute to the support of tumor growth and targeted to prevent tumor recurrence. The ability of relacorilant to induce PitNET cell death in a multicellular culture system is the first step in the development of effective targeted therapies for patients with CD and potentially, other PitNET subtypes.

### REFERENCES

[1] H. Cushing, Posterior Pituitary Activity from an Anatomical Standpoint, Am J Pathol, 9 (1933) 539-548 519.
[2] H. Cushing, The basophil adenomas of the pituitary body and their clinical manifestations (pituitary basophilism). 1932, Obes Res, 2 (1994) 486-508.
[3] N. Ironside, C. J. Chen, C. C. Lee, D. M. Trifiletti, M. L. Vance, J. P. Sheehan, Outcomes of Pituitary Radiation for Cushing's Disease, Endocrinol Metab Clin North Am, 47 (2018) 349-365.
[4] D. L. Loriaux, Diagnosis and Differential Diagnosis of Cushing's Syndrome, N Engl J Med, 377 (2017) e3.
[5] S. L. Asa, O. Mete, A. Perry, R. Y. Osamura, Overview of the 2022 WHO Classification of Pituitary Tumors, Endocr Pathol, 33 (2022) 6-26.
[6] H. Nishioka, S. Yamada, Cushing's Disease, J Clin Med, 8 (2019).
[7] R. A. Feelders, L. J. Hofland, Medical treatment of Cushing's disease, J Clin Endocrinol Metab, 98 (2013) 425-438.
[8] P. Limumpornpetch, A. W. Morgan, A. Tiganescu, P. D. Baxter, V. Nyawira Nyaga, M. Pujades-Rodriguez, P. M. Stewart, The Effect of Endogenous Cushing Syndrome on All-cause and Cause-specific Mortality, J Clin Endocrinol Metab, 107 (2022) 2377-2388.
[9] A. B. Atkinson, A. Kennedy, M. I. Wiggam, D. R. McCance, B. Sheridan, Long-term remission rates after pituitary surgery for Cushing's disease: the need for long-term surveillance, Clin Endocrinol (Oxf), 63 (2005) 549-559.
[10] R. van der Pas, R. A. Feelders, F. Gatto, C. de Bruin, A. M. Pereira, P. M. van Koetsveld, D. M. Sprij-Mooij, A. M. Waaijers, F. Dogan, S. Schulz, J. M. Kros, S. W. Lamberts, L. J. Hofland, Preoperative normalization of cortisol levels in Cushing's disease after medical treatment: consequences for somatostatin and dopamine receptor subtype expression and in vitro response to somatostatin analogs and dopamine agonists, J Clin Endocrinol Metab, 98 (2013) E1880-1890.
[11] D. Kondziolka, Cushing's disease and stereotactic radiosurgery, J Neurosurg, 119 (2013) 1484-1485; discussion 1485.
[12] G. U. Mehta, J. P. Sheehan, M. L. Vance, Effect of stereotactic radiosurgery before bilateral adrenalectomy for Cushing's disease on the incidence of Nelson's syndrome, J Neurosurg, 119 (2013) 1493-1497.
[13] M. Fleseriu, B. M. Biller, J. W. Findling, M. E. Molitch, D. E. Schteingart, C. Gross, S. S. Investigators, mifepristone, a glucocorticoid receptor antagonist, produces clinical and metabolic benefits in patients with Cushing's syndrome, J Clin Endocrinol Metab, 97 (2012) 2039-2049.
[14] J. Feiteiro, M. Mariana, I. Verde, E. Cairrao, Genomic and Nongenomic Effects of Mifepristone at the Cardiovascular Level: A Review, Reprod Sci, 24 (2017) 976-988.
[15] H. Hunt, K. Donaldson, M. Strem, V. Zann, P. Leung, S. Sweet, A. Connor, D. Combs, J. Belanoff, Assessment of Safety, Tolerability, Pharmacokinetics, and Pharmacological Effect of Orally Administered CORT125134: An Adaptive, Double-Blind, Randomized, Placebo-Controlled Phase 1 Clinical Study, Clin Pharmacol Drug Dev, 7 (2018) 408-421.
[16] H. J. Hunt, J. K. Belanoff, I. Walters, B. Gourdet, J. Thomas, N. Barton, J. Unitt, T. Phillips, D. Swift, E. Eaton, Identification of the Clinical Candidate (R)-(1-(4-Fluorophenyl)-6-((1-methyl-1H-pyrazol-4-yl) sulfonyl)-4,4a,5,6,7,8-hexahydro-1H-pyrazolo[3,4-g]isoquinolin-4a-yl) (4-(trifluoromethyl)pyridin-2-yl) methano ne (CORT125134): A Selective Glucocorticoid Receptor (G R) Antagonist, J Med Chem, 60 (2017) 3405-3421.
[17] M. Massimo Terzolo, Davide Iacuaniello, M D, Anna Pia, M D, Priola Adriano, M D, Andreas Moraitis, M D, and Rosario Pivonello, M D, SUN-463 Tumor Shrinkage with Preoperative Relacorilant Therapy in Two Patients with Cushing Disease Due to Pituitary Macroadenomas, Journal of the Endocrine Society, (2019).
[18] R. Pivonello, P. N. Munster, M. Terzolo, R. Ferrigno, C. Simeoli, S. Puglisi, U. Bali, A. G. Moraitis, Glucocorticoid Receptor Antagonism Upregulates Somatostatin Receptor Subtype 2 Expression in ACTH-Producing Neuroendocrine Tumors: New Insight Based on the Selective Glucocorticoid Receptor Modulator Relacorilant, Front Endocrinol (Lausanne), 12 (2021) 793262.
[19] R. Pivonello, R. Ferrigno, M. C. De Martino, C. Simeoli, N. Di Paola, C. Pivonello, L. Barba, M. Negri, C. De Angelis, A. Colao, Medical Treatment of Cushing's Disease: An Overview of the Current and Recent Clinical Trials, Front Endocrinol (Lausanne), 11 (2020) 648.
[20] F. Gatto, F. Barbieri, M. Arvigo, S. Thellung, J. Amaru, M. Albertelli, D. Ferone, T. Florio, Biological and Biochemical Basis of the Differential Efficacy of First and Second Generation Somatostatin Receptor Ligands in Neuroendocrine Neoplasms, Int J Mol Sci, 20 (2019).
[21] R. A. Feelders, C. de Bruin, A. M. Pereira, J. A. Romijn, R. T. Netea-Maier, A. R. Hermus, P. M. Zelissen, R. van Heerebeek, F. H. de Jong, A. J. van der Lely, W. W. de Herder, L. J. Hofland, S. W. Lamberts, Pasireotide alone or with cabergoline and ketoconazole in Cushing's disease, N Engl J Med, 362 (2010) 1846-1848.
[22] A. Lacroix, F. Gu, W. Gallardo, R. Pivonello, Y. Yu, P. Witek, M. Boscaro, R. Salvatori, M. Yamada, L. Tauchmanova, M. Roughton, S. Ravichandran, S. Petersenn, B. M. K. Biller, J. Newell-Price, G. S. G. Pasireotide, Efficacy and safety of once-monthly pasireotide in Cushing's disease: a 12 month clinical trial, Lancet Diabetes Endocrinol, 6 (2018) 17-26.
[23] A. Colao, S. Petersenn, J. Newell-Price, J. W. Findling, F. Gu, M. Maldonado, U. Schoenherr, D. Mills, L. R. Salgado, B. M. Biller, B. S. G. Pasireotide, A 12-month phase 3 study of pasireotide in Cushing's disease, N Engl J Med, 366 (2012) 914-924.
[24] K. H. Narsinh, F. Jia, R. C. Robbins, M. A. Kay, M. T. Longaker, J. C. Wu, Generation of adult human induced pluripotent stem cells using nonviral minicircle DNA vectors, Nat Protoc, 6 (2011) 78-88.
[25] C. A. Stratakis, M. A. Tichomirowa, S. Boikos, M. F. Azevedo, M. Lodish, M. Martari, S. Verma, A. F. Daly, M. Raygada, M. F. Keil, J. Papademetriou, L. Drori-Herishanu, A. Horvath, K. M. Tsang, M. Nesterova, S. Franklin, J. F. Vanbellinghen, V. Bours, R. Salvatori, A. Beckers, The role of germline AIP, MEN1, PRKAR1A, CDKN1B and CDKN2C mutations in causing pituitary adenomas in a large cohort of children, adolescents, and patients with genetic syndromes, Clin Genet, 78 (2010) 457-463.
[26] Z. Y. Ma, Z. J. Song, J. H. Chen, Y. F. Wang, S. Q. Li, L. F. Zhou, Y. Mao, Y. M. Li, R. G. Hu, Z. Y. Zhang, H. Y. Ye, M. Shen, X. F. Shou, Z. Q. Li, H. Peng, Q. Z. Wang, D. Z. Zhou, X. L. Qin, J. Ji, J. Zheng, H. Chen, Y. Wang, D. Y. Geng, W. J. Tang, C. W. Fu, Z. F. Shi, Y. C. Zhang, Z. Ye, W. Q. He, Q. L. Zhang, Q. S. Tang, R. Xie, J. W. Shen, Z. J. Wen, J. Zhou, T. Wang, S. Huang, H. J. Qiu, N. D. Qiao, Y. Zhang, L. Pan, W. M. Bao, Y. C. Liu, C. X. Huang, Y. Y. Shi, Y. Zhao, Recurrent gain-of-function USP8 mutations in Cushing's disease, Cell Res, 25 (2015) 306-317.
[27] M. Reincke, S. Sbiera, A. Hayakawa, M. Theodoropoulou, A. Osswald, F. Beuschlein, T. Meitinger, E. Mizuno-Yamasaki, K. Kawaguchi, Y. Saeki, K. Tanaka, T. Wieland, E. Graf, W. Saeger, C. L. Ronchi, B. Allolio, M. Buchfelder, T. M. Strom, M. Fassnacht, M. Komada, Mutations in the deubiquitinase gene USP8 cause Cushing's disease, Nat Genet, 47 (2015) 31-38.
[28] M. Reincke, M. Theodoropoulou, Genomics in Cushing's Disease: The Dawn of a New Era, J Clin Endocrinol Metab, 106 (2021) e2455-e2456.
[29] N. G. Steele, J. Chakrabarti, J. Wang, J. Biesiada, L. Holokai, J. Chang, L. M. Nowacki, J. Hawkins, M. Mahe, N. Sundaram, N. Shroyer, M. Medvedovic, M. Helmrath, S. Ahmad, Y. Zavros, An Organoid-Based Preclinical Model of Human Gastric Cancer, Cell Mol Gastroenterol Hepatol, 7 (2019) 161-184.
[30] R. Feng, E. Aihara, S. Kenny, L. Yang, J. Li, A. Varro, M. H. Montrose, N. F. Shroyer, T. C. Wang, R. A. Shivdasani, Y. Zavros, Indian Hedgehog mediates gastrin-induced proliferation in stomach of adult mice, Gastroenterology, 147 (2014) 655-666 e659.
[31] E. C. Filippi-Chiela, M. M. Oliveira, B. Jurkovski, S. M. Callegari-Jacques, V. D. da Silva, G. Lenz, Nuclear morphometric analysis (NMA): screening of senescence, apoptosis and nuclear irregularities, PLOS One, 7 (2012) e42522.
[32] K. J. Livak, T. D. Schmittgen, Analysis of relative gene expression data using real-time quantitative PCR and the 2(-Delta Delta C (T)) Method, Methods, 25 (2001) 402-408.
[33] J. Chen, X. Jian, S. Deng, Z. Ma, X. Shou, Y. Shen, Q. Zhang, Z. Song, Z. Li, H. Peng, C. Peng, M. Chen, C. Luo, D. Zhao, Z. Ye, M. Shen, Y. Zhang, J. Zhou, A. Fahira, Y. Wang, S. Li, Z. Zhang, H. Ye, Y. Li, J. Shen, H. Chen, F. Tang, Z. Yao, Z. Shi, C. Chen, L. Xie, Y. Wang, C. Fu, Y. Mao, L. Zhou, D. Gao, H. Yan, Y. Zhao, C. Huang, Y. Shi, Identification of recurrent USP48 and BRAF mutations in Cushing's disease, Nat Commun, 9 (2018) 3171.
[34] B. Zimmer, J. Piao, K. Ramnarine, M. J. Tomishima, V. Tabar, L. Studer, Derivation of Diverse Hormone-Releasing Pituitary Cells from Human Pluripotent Stem Cells, Stem Cell Reports, 6 (2016) 858-872.
[35] X. Zhu, A. S. Gleiberman, M. G. Rosenfeld, Molecular physiology of pituitary development: signaling and transcriptional networks, Physiol Rev, 87 (2007) 933-963.
[36] C. Ballmann, A. Thiel, H. E. Korah, A. C. Reis, W. Saeger, S. Stepanow, K. Kohrer, G. Reifenberger, C. B. Knobbe-Thomsen, U. J. Knappe, U. I. Scholl, USP8 Mutations in Pituitary Cushing Adenomas-Targeted Analysis by Next-Generation Sequencing, J Endocr Soc, 2 (2018) 266-278.
[37] T. Sahakitrungruang, C. Srichomthong, S. Pornkunwilai, J. Amornfa, S. Shuangshoti, S. Kulawonganunchai, K. Suphapeetiporn, V. Shotelersuk, Germline and somatic DICERI mutations in a pituitary blastoma causing infantile-onset Cushing's disease, J Clin Endocrinol Metab, 99 (2014) E1487-1492.
[38] F. Cetani, E. Pardi, L. Cianferotti, E. Vignali, A. Picone, P. Miccoli, A. Pinchera, C. Marcocci, A new mutation of the MEN1 gene in an italian kindred with multiple endocrine neoplasia type 1, Eur J Endocrinol, 140 (1999) 429-433.
[39] F. Marini, F. Giusti, C. Fossi, F. Cioppi, L. Cianferotti, L. Masi, F. Boaretto, S. Zovato, F. Cetani, A. Colao, M. V. Davi, A. Faggiano, G. Fanciulli, P. Ferolla, D. Ferone, P. Loli, F. Mantero, C. Marcocci, G. Opocher, P. Beck-Peccoz, L. Persani, A. Scillitani, F. Guizzardi, A. Spada, P. Tomassetti, F. Tonelli, M. L. Brandi, Multiple endocrine neoplasia type 1: analysis of germline MEN1 mutations in the Italian multicenter MEN1 patient database, Endocrine, 62 (2018) 215-233.
[40] L. Cazabat, J. Bouligand, P. Chanson, AIP mutation in pituitary adenomas, N Engl J Med, 364 (2011) 1973-1974; author reply 1974-1975.
[41] L. Cazabat, J. Bouligand, S. Salenave, M. Bernier, S. Gaillard, F. Parker, J. Young, A. Guiochon-Mantel, P. Chanson, Germline AIP mutations in apparently sporadic pituitary adenomas: prevalence in a prospective single-center cohort of 443 patients, J Clin Endocrinol Metab, 97 (2012) E663-670.
[42] D. Brennan, X. Chen, L. Cheng, M. Mahoney, N. A. Riobo, Noncanonical Hedgehog signaling, Vitam Horm, 88 (2012) 55-72.
[43] R. Teperino, F. Aberger, H. Esterbauer, N. Riobo, J. A. Pospisilik, Canonical and non-canonical Hedgehog signalling and the control of metabolism, Semin Cell Dev Biol, 33 (2014) 81-92.
[44] D. Bongiovanni, V. Tosello, V. Saccomani, S. Dalla Santa, A. Amadori, P. Zanovello, E. Piovan, Crosstalk between Hedgehog pathway and the glucocorticoid receptor pathway as a basis for combination therapy in T-cell acute lymphoblastic leukemia, Oncogene, 39 (2020) 6544-6555.
[45] C. Xiao, R. Feng, A. Engevik, J. Martin, J. Tritschler, M. Schumacher, R. Koncar, J. Roland, K. Nam, J. Goldenring, Y. Zavros, Sonic Hedgehog contributes to gastric mucosal restitution after injury., Lab Invest, 93 (2013) 96-111.
[46] C. Xiao, S. A. Ogle, M. A. Schumacher, M. A. Orr-Asman, M. L. Miller, N. Lertkowit, A. Varro, F. Hollande, Y. Zavros, Loss of Parietal Cell Expression of Sonic Hedgehog Induces Hypergastrinemia and Hyperproliferation of Surface Mucous Cells., Gastroenterology, 138 (2010) 550-561.
[47] M. Treier, S. O'Connell, A. Gleiberman, J. Price, D. P. Szeto, R. Burgess, P. T. Chuang, A. P. McMahon, M. G. Rosenfeld, Hedgehog signaling is required for pituitary gland development, Development, 128 (2001) 377-386.
[48] M. Fleseriu, J. W. Findling, C. A. Koch, S. M. Schlaffer, M. Buchfelder, C. Gross, Changes in plasma ACTH levels and corticotroph tumor size in patients with Cushing's disease during long-term treatment with the glucocorticoid receptor antagonist mifepristone, J Clin Endocrinol Metab, 99 (2014) 3718-3727.
[49] M. Grant, H. Alturaihi, P. Jaquet, B. Collier, U. Kumar, Cell growth inhibition and functioning of human somatostatin receptor type 2 are modulated by receptor heterodimerization, Mol Endocrinol, 22 (2008) 2278-2292.
[50] M. Cakir, D. Dworakowska, A. Grossman, Somatostatin receptor biology in neuroendocrine and pituitary tumours: part 1—molecular pathways, J Cell Mol Med, 14 (2010) 2570-2584.
[51] N. Sharif, L. Gendron, J. Wowchuk, P. Sarret, J. Mazella, A. Beaudet, T. Stroh, Coexpression of somatostatin receptor subtype 5 affects internalization and trafficking of somatostatin receptor subtype 2, Endocrinology, 148 (2007) 2095-2105.
[52] N. Ahmad, R. Kumar, Steroid hormone receptors in cancer development: a target for cancer therapeutics, Cancer Lett, 300 (2011) 1-9.
[53] P. N. Munster, A. E. Greenstein, G. F. Fleming, E. Borazanci, M. R. Sharma, J. M. Custodio, I. C. Tudor, H. I. Pashova, S. Peacock Shepherd, A. Grauer, J. C. Sachdev, Overcoming Taxane Resistance: Preclinical and Phase 1 Studies of Relacorilant, a Selective Glucocorticoid Receptor Antagonist, with Nab-Paclitaxel in Solid Tumors, Clin Cancer Res, (2022).
[54] A. E. Greenstein, H. J. Hunt, Glucocorticoid receptor antagonism promotes apoptosis in solid tumor cells, Oncotarget, 12 (2021) 1243-1255.
[55] A. L. Gruver-Yates, J. A. Cidlowski, Tissue-specific actions of glucocorticoids on apoptosis: a double-edged sword, Cells, 2 (2013) 202-223.
[56] L. Holokai, J. Chakrabarti, J. Lundy, D. Croagh, P. Adhikary, S. S. Richards, C. Woodson, N. Steele, R. Kuester, A. Scott, M. Khreiss, T. Frankel, J. Merchant, B. J. Jenkins, J. Wang, R. T. Shroff, S. A. Ahmad, Y. Zavros, Murine- and Human-Derived Autologous Organoid/Immune Cell Co-Cultures as Pre-Clinical Models of Pancreatic Ductal Adenocarcinoma, Cancers (Basel), 12 (2020).
[57] S. F. Boj, C. I. Hwang, L. A. Baker, Chio, I I, D. D. Engle, V. Corbo, M. Jager, M. Ponz-Sarvise, H. Tiriac, M. S. Spector, A. Gracanin, T. Oni, K. H. Yu, R. van Boxtel, M. Huch, K. D. Rivera, J. P. Wilson, M. E. Feigin, D. Ohlund, A. Handly-Santana, C. M. Ardito-Abraham, M. Ludwig, E. Elyada, B. Alagesan, G. Biffi, G. N. Yordanov, B. Delcuze, B. Creighton, K. Wright, Y. Park, F. H. Morsink, I. Q. Molenaar, I. H. Borel Rinkes, E. Cuppen, Y. Hao, Y. Jin, I. J. Nijman, C. Iacobuzio-Donahue, S. D. Leach, D. J. Pappin, M. Hammell, D. S. Klimstra, O. Basturk, R. H. Hruban, G. J. Offerhaus, R. G. Vries, H. Clevers, D. A. Tuveson, Organoid models of human and mouse ductal pancreatic cancer, Cell, 160 (2015) 324-338.
[58] H. Tiriac, P. Belleau, D. D. Engle, D. Plenker, A. Deschenes, T. D. D. Somerville, F. E. M. Froeling, R. A. Burkhart, R. E. Denroche, G. H. Jang, K. Miyabayashi, C. M. Young, H. Patel, M. Ma, J. F. LaComb, R. L. D. Palmaira, A. A. Javed, J. C. Huynh, M. Johnson, K. Arora, N. Robine, M. Shah, R. Sanghvi, A. B. Goetz, C. Y. Lowder, L. Martello, E. Driehuis, N. LeComte, G. Askan, C. A. Iacobuzio-Donahue, H. Clevers, L. D. Wood, R. H. Hruban, E. Thompson, A. J. Aguirre, B. M. Wolpin, A. Sasson, J. Kim, M. Wu, J. C. Bucobo, P. Allen, D. V. Sejpal, W. Nealon, J. D. Sullivan, J. M. Winter, P. A. Gimotty, J. L. Grem, D. J. DiMaio, J. M. Buscaglia, P. M. Grandgenett, J. R. Brody, M. A. Hollingsworth, G. M. O'Kane, F. Notta, E. Kim, J. M. Crawford, C. Devoe, A. Ocean, C. L. Wolfgang, K. H. Yu, E. Li, C. R. Vakoc, B. Hubert, S. E. Fischer, J. M. Wilson, R. Moffitt, J. Knox, A. Krasnitz, S. Gallinger, D. A. Tuveson, Organoid Profiling Identifies Common Responders to Chemotherapy in Pancreatic Cancer, Cancer Discov, 8 (2018) 1112-1129.
[59] D. C. Danila, X. Zhang, Y. Zhou, G. R. Dickersin, J. A. Fletcher, E. T. Hedley-Whyte, M. K. Selig, S. R. Johnson, A. Klibanski, A human pituitary tumor-derived folliculostellate cell line, J Clin Endocrinol Metab, 85 (2000) 1180-1187.
[60] T. Bjoro, P. A. Torjesen, B. C. Ostberg, O. Sand, J. G. Iversen, K. M. Gautvik, E. Haug, Bombesin stimulates prolactin secretion from cultured rat pituitary tumour cells (GH4C1) via activation of phospholipase C, Regul Pept, 19 (1987) 169-182.
[61] T. Bjoro, O. Sand, B. C. Ostberg, J. O. Gordeladze, P. Torjesen, K. M. Gautvik, E. Haug, The mechanisms by which vasoactive intestinal peptide (VIP) and thyrotropin releasing hormone (TRH) stimulate prolactin release from pituitary cells, Biosci Rep, 10 (1990) 189-199.
[62] H. Ikeda, T. Mitsuhashi, K. Kubota, N. Kuzuya, H. Uchimura, Epidermal growth factor stimulates growth hormone secretion from superfused rat adenohypophyseal fragments, Endocrinology, 115 (1984) 556-558.
[63] N. Baek, O. W. Seo, M. Kim, J. Hulme, S. S. An, Monitoring the effects of doxorubicin on 3D-spheroid tumor cells in real-time, Onco Targets Ther, 9 (2016) 7207-7218.
[64] B. Cox, E. Laporte, A. Vennekens, H. Kobayashi, C. Nys, I. Van Zundert, I. H. Uji, A. Vercauteren Drubbel, B. Beck, H. Roose, M. Boretto, H. Vankelecom, Organoids from pituitary as a novel research model toward pituitary stem cell exploration, J Endocrinol, 240 (2019) 287-308.
[65] A. Vennekens, E. Laporte, F. Hermans, B. Cox, E. Modave, A. Janiszewski, C. Nys, H. Kobayashi, B. Malengier-Devlies, J. Chappell, P. Matthys, M. I. Garcia, V. Pasque, D. Lambrechts, H. Vankelecom, Interleukin-6 is an activator of pituitary stem cells upon local damage, a competence quenched in the aging gland, Proc Natl Acad Sci USA, 118 (2021).
[66] E. Laporte, C. Nys, H. Vankelecom, Development of Organoids from Mouse Pituitary as In Vitro Model to Explore Pituitary Stem Cell Biology, J Vis Exp, (2022).
[67] C. Nys, Y. L. Lee, H. Roose, F. Mertens, E. De Pauw, H. Kobayashi, R. Sciot, M. Bex, G. Versyck, S. De Vleeschouwer, J. Van Loon, E. Laporte, H. Vankelecom, Exploring stem cell biology in pituitary tumors and derived organoids, Endocr Relat Cancer, 29 (2022) 427-450.
[68] https://www.cancer.gov/publications/dictionaries/cancer-terms/def/organoid.
[69] A. Shintani, M. Higuchi, Isolation of PRRX1-positive adult pituitary stem/progenitor cells from the marginal cell layer of the mouse anterior lobe, Stem Cell Res, 52 (2021) 102223.
[70] R. Wurth, F. Barbieri, A. Pattarozzi, G. Gaudenzi, F. Gatto, P. Fiaschi, J. L. Ravetti, G. Zona, A. Daga, L. Persani, D. Ferone, G. Vitale, T. Florio, Phenotypical and Pharmacological Characterization of Stem-Like Cells in Human Pituitary Adenomas, Mol Neurobiol, 54 (2017) 4879-4895.
[71] R. Wurth, S. Thellung, A. Corsaro, F. Barbieri, T. Florio, Experimental Evidence and Clinical Implications of Pituitary Adenoma Stem Cells, Front Endocrinol (Lausanne), 11 (2020) 54.
[72] T. Kasai, H. Suga, M. Sakakibara, C. Ozone, R. Matsumoto, M. Kano, K. Mitsumoto, K. Ogawa, Y. Kodani, H. Nagasaki, N. Inoshita, M. Sugiyama, T. Onoue, T. Tsunekawa, Y. Ito, H. Takagi, D. Hagiwara, S. Iwama, M. Goto, R. Banno, J. Takahashi, H. Arima, Hypothalamic Contribution to Pituitary Functions Is Recapitulated In Vitro Using 3D-Cultured Human iPS Cells, Cell Rep, 30 (2020) 18-24 e15.
[73] G. Mantovani, E. Giardino, D. Treppiedi, R. Catalano, F. Mangili, A. Spada, M. Arosio, E. Peverelli, Stem Cells in Pituitary Tumors: Experimental Evidence Supporting Their Existence and Their Role in Tumor Clinical Behavior, Front Endocrinol (Lausanne), 10 (2019) 745.
[74] Y. Zhou, R. R. A. Wilson, A. Udaiyar, J. Mclemore, H. Sadri-Ardekani, T. Criswell, Pituitary lineage differentiation from human-induced pluripotent stem cells in 2D and 3D cultures, Stem Cells Dev, (2022).
[75] D. Zhang, W. Hugo, P. Redublo, H. Miao, M. Bergsneider, M. B. Wang, W. Kim, W. H. Yong, A. P. Heaney, A human ACTH-secreting corticotroph tumoroid model: Novel Human ACTH-Secreting Tumor Cell in vitro Model, EBioMedicine, 66 (2021) 103294.

1-50. (canceled)

51. A method of treating Cushing's syndrome, comprising administration of a non-steroidal selective glucocorticoid receptor modulator (non-steroidal SGRM) for selectively increasing SSTR5 expression in human pituitary cells, wherein said non-steroidal SGRM is a is a heteroaryl-ketone fused azadecalin compound capable of selectively stimulating the expression of SSTR5, whereby SSTR5 expression in said human pituitary cells is selectively increased and said Cushing's syndrome is treated.

52. The method of claim 51, wherein the non-steroidal SGRM is the heteroaryl-ketone fused azadecalin compound (R)-(1-(4-fluorophenyl)-6-((1-methyl-1H-pyrazol-4-yl) sulfonyl)-4,4a,5,6,7,8-hexahydro-1H-pyrazolo[-3,4-g]isoquinolin-4a-yl) (4-(trifluoromethyl)pyridin-2-yl) methanone (relacorilant) having the structure:

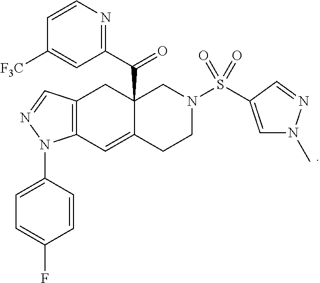 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

53. The method of claim 51, comprising administration of a somatostatin or somatostatin analog which binds to SSTR5.

54. The method of claim 52, comprising administration of a somatostatin or somatostatin analog which binds to SSTR5.

55. The method of claim 53, comprising administration of the somatostatin analog pasireotide.

56. The method of claim 54, comprising administration of the somatostatin analog pasireotide.

57. A method of selectively increasing SSTR5 expression in human pituitary cells for treating a pituitary tumor without significantly increasing the level of ACTH, the method comprising administration of a non-steroidal selective glucocorticoid receptor modulator (non-steroidal SGRM), wherein said non-steroidal SGRM is a heteroaryl-ketone fused azadecalin compound capable of selectively stimulating the expression of SSTR5,
whereby SSTR5 expression in said human pituitary cells is selectively increased without significantly increasing the level of ACTH and said Cushing's syndrome is treated.

58. The method of claim 57, wherein the non-steroidal SGRM is the heteroaryl-ketone fused azadecalin compound (R)-(1-(4-fluorophenyl)-6-((1-methyl-1H-pyrazol-4-yl) sulfonyl)-4,4a,5,6,7,8-hexahydro-1H-pyrazolo[-3,4-g]isoquinolin-4a-yl) (4-(trifluoromethyl)pyridin-2-yl) methanone (relacorilant) having the structure:

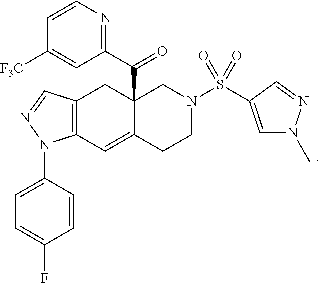 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

59. The method of claim 57, comprising administration of a somatostatin or somatostatin analog which binds to SSTR5.

60. The method of claim 58, comprising administration of a somatostatin or somatostatin analog which binds to SSTR5.

61. The method of claim 59, comprising administration of the somatostatin analog pasireotide.

62. The method of claim 60, comprising administration of the somatostatin analog pasireotide.

63. A method of treating Cushing's syndrome, comprising administration of a non-steroidal selective glucocorticoid receptor modulator (non-steroidal SGRM) for selectively increasing SSTR5 expression in human pituitary cells for inducing apoptosis in a pituitary tumor without significantly increasing the level of ACTH by administration of a non-steroidal selective glucocorticoid receptor modulator (non-steroidal SGRM), wherein the non-steroidal SGRM is a heteroaryl-ketone fused azadecalin compound capable of selectively stimulating the expression of SSTR5, whereby SSTR5 expression is selectively increased and apoptosis is induced in human pituitary cells in a pituitary tumor without significantly increasing the level of ACTH, and said Cushing's syndrome is treated.

64. The method of claim 63, wherein the non-steroidal SGRM is the heteroaryl-ketone fused azadecalin compound (R)-(1-(4-fluorophenyl)-6-((1-methyl-1H-pyrazol-4-yl) sulfonyl)-4,4a,5,6,7,8-hexahydro-1H-pyrazolo[-3,4-g]isoquinolin-4a-yl) (4-(trifluoromethyl)pyridin-2-yl) methanone (relacorilant) having the structure:

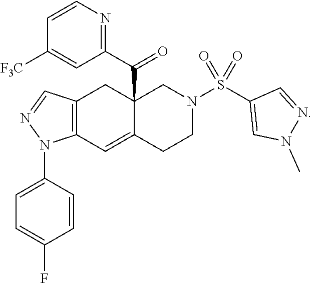 [SMILES: Cn1cc(S(=O)(=O)N2CCC3=Cc4c(cnn4-c4ccc(F)cc4)C[C@]3(C(=O)c3cc(C(F)(F)F)ccn3)C2)cn1]

65. The method of claim 63, comprising administration of a somatostatin or somatostatin analog which binds to SSTR5.

66. The method of claim 64, comprising administration of a somatostatin or somatostatin analog which binds to SSTR5.

67. The method of claim 65, comprising administration of the somatostatin analog pasireotide.

68. The method of claim 66, comprising administration of the somatostatin analog pasireotide.
